(19) United States
(12) Patent Application Publication
(10) Pub. No.: US 2023/0304860 A1
(43) Pub. Date: Sep. 28, 2023

(54) GENERALIZED ARTIFICIAL INTELLIGENCE MODELER FOR ULTRA-WIDE-SCALE DEPLOYMENT OF SPECTRAL DEVICES

(71) Applicant: Si-Ware Systems, Cairo (EG)

(72) Inventors: Yasser M. Sabry, Cairo (EG); Bassem Mortada, Cairo (EG); Samir Abozyd, Tanta (EG); Mostafa Medhat, Cairo (EG); Mai Said, Cairo (EG); Bassam Saadany, Cairo (EG); Youssri Helmy, Los Altos Hills, CA (US); Ahmed Badr, Allendale, NJ (US); Marcal Plans Pujolras, Llinars del Valles (ES)

(21) Appl. No.: 18/124,912

(22) Filed: Mar. 22, 2023

Related U.S. Application Data
(60) Provisional application No. 63/323,036, filed on Mar. 23, 2022.

(51) Int. Cl. G01J 3/02; G06N 20/00; G01J 3/45

(52) U.S. Cl. CPC G01J 3/0297; G06N 20/00; G01J 3/45; G01J 2003/2879

(57) ABSTRACT

Aspects relate to a spectral modeling system for building chemometrics (calibration) models for spectral devices targeting ultra-wide-scale deployment. The spectral modeling system includes a spectral converter for generating a plurality of artificial spectra using spectral data of a plurality of samples measured by a subset of a plurality of spectral devices and spectral device characteristics representing spectral variations in the plurality of spectral devices. The spectral modeling system further includes a chemometrics engine for generating a chemometrics model for one or more parameters associated with the plurality of samples based on the spectral data and the plurality of artificial spectra.

### PRIORITY CLAIM

This application claims priority to and the benefit of Provisional Application No. 63/323,036, filed in the U.S. Patent and Trademark Office on Mar. 23, 2022, the entire content of which is incorporated herein by reference as if fully set forth below in its entirety and for all applicable purposes.

### TECHNICAL FIELD

The technology discussed below relates generally to artificial intelligence modelers for building chemometrics models for spectral devices, and more particular to mechanisms for building generalized chemometrics models targeting ultra-wide-scale deployment of spectral devices.

### BACKGROUND

In spectral sensing, the interaction between electromagnetic radiation, such as light, and matter is studied. There are different types of spectroscopy used, such as infrared/vibrational spectroscopy, atomic absorption spectroscopy, mass spectroscopy, electrochemical impedance spectroscopy, x-ray spectroscopy, in addition to others. The development of analytical chemistry devices based on infrared spectral sensing devices have progressed quickly in the last decade. The development went through a paradigm shift moving from laboratory-based bench top devices to handheld devices that can be used in the field or in-line in the production facilities in a ubiquitous manner. The mid infrared (MIR) wavelength range (2.5 μm to 25 μm) contains spectral lines corresponding to fundamental vibrations lines. The IR range below 2.5 μm is the near infrared (NIR) range that includes the overtones and the combinational lines. With the development in multivariate statistical methods, called chemometrics, qualitative and quantitative material analysis is possible using the infrared spectra.

### SUMMARY

The following presents a summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a form as a prelude to the more detailed description that is presented later.

In an example, a spectral modeling system is disclosed. The spectral modeling system includes a spectral converter configured to receive spectral data of a plurality of samples from a subset of a plurality of spectral devices and spectral device characteristics representing spectral variations in the plurality of spectral devices. The spectral converter is further configured to generate a plurality of artificial spectra representing remaining spectral devices of the plurality of spectral devices based on the spectral data and the spectral device characteristics. The spectral modeling system further includes a chemometrics engine configured to produce a chemometrics model for one or more parameters associated with the plurality of samples based on the spectral data and the plurality of artificial spectra.

Another example provides a method for spectral modeling. The method includes receiving spectral data of a plurality of samples from a subset of a plurality of spectral devices, receiving spectral device characteristics representing spectral variations in the plurality of spectral devices, and generating a plurality of artificial spectra representing remaining spectral devices of the plurality of spectral devices based on the spectral data and the spectral device characteristics. The method further includes producing a chemometrics model for one or more parameters associated with the plurality of samples based on the spectral data and the plurality of artificial spectra.

These and other aspects of the invention will become more fully understood upon a review of the detailed description, which follows. Other aspects, features, and embodiments of the present invention will become apparent to those of ordinary skill in the art, upon reviewing the following description of specific, exemplary embodiments of the present invention in conjunction with the accompanying figures. While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with the various embodiments of the invention discussed herein. In similar fashion, while exemplary embodiments may be discussed below as device, system, or method embodiments it should be understood that such exemplary embodiments can be implemented in various devices, systems, and methods.

### BRIEF DESCRIPTION OF THE DRAWINGS

FIG. 1 is a diagram illustrating an example of a spectrometer according to some aspects.

FIG. 2 is a diagram illustrating another example of a spectrometer according to some aspect.

FIG. 3 is a diagram illustrating another example of a spectrometer according to some aspects.

FIG. 4 is a diagram illustrating an example of a chemometrics model according to some aspects.

FIGS. 5A-5E are graphs illustrating various specifications of spectral devices according to some aspects.

FIGS. 6A and 6B are graphs illustrating variations in self-apodization of a spectral device and corresponding spectral variations according to some aspects.

FIG. 7 is a diagram illustrating an example of a spectral modeling system according to some aspects.

FIG. 8 is a diagram illustrating an example of a spectral modeling system for two spectral devices and according to some aspects.

FIG. 9 is a diagram illustrating an example of a characteristics extractor according to some aspects.

FIGS. 10A and 10B illustrate an example of a spectral device configured for Stage 1 characteristic extraction according to some aspects.

FIG. 11A-11D illustrate an example of a spectral device configured for Stage 2 or Stage 3 characteristic extraction according to some aspects.

FIG. 12 is a diagram illustrating an example extractor setup for Stage 4 according to some aspects.

FIG. 13 is a diagram illustrating another example of a spectral modeling system for two spectral devices and according to some aspects.

FIGS. 14A and 14B are diagrams illustrating an example of a global spectral converter according to some aspects.

FIGS. 15A and 15B are graphs representing the impact on the bias and slope errors in the prediction according to some aspects.

FIG. 16 is a diagram illustrating another example of a global spectral converter according to some aspects.

FIG. 17 is a diagram illustrating another example of a global spectral converter according to some aspects.

FIG. 18 is a diagram illustrating another example of a global spectral converter according to some aspects.

FIGS. 19A-19D are diagrams illustrating examples of pre-processing of the spectral data according to some aspects.

FIG. 20 is a diagram illustrating another example of a global spectral converter according to some aspects.

FIGS. 21A-21C are diagrams illustrating an example of spectral correction according to some aspects.

FIG. 22 is a diagram illustrating an example of an algorithm for selection of the subset of spectral devices to collect the spectral data according to some aspects.

FIG. 23 is a diagram illustrating another example of spectral correction according to some aspects.

FIG. 24 is a diagram illustrating another example of spectral correction according to some aspects.

FIG. 25 is a diagram illustrating examples of optical head configuration generalization according to some examples.

FIG. 26 is a diagram illustrating another example of spectral correction according to some aspects.

FIG. 27 is a diagram illustrating an example of a process flow for generating artificial spectra according to some aspects.

FIGS. 28A-28C illustrate an example of generalization of spectral data based on self-apodization and resolution variations according to some aspects.

FIGS. 29A-29C illustrate an example of generalization of spectral data based on wavenumber/wavelength errors according to some aspects.

FIGS. 30A-30C illustrate an example of generalization of spectral data based on SNR across the wavelength range according to some aspects.

FIGS. 31A-31C illustrate an example of generalization of spectral data based on absorbance scaling according to some aspects.

FIGS. 32A and 32B illustrate an example of generalization of spectral data based on thermal drift according to some aspects.

FIGS. 33A and 33B illustrate an example of generalization of spectral data based on baseline shift according to some aspects.

FIGS. 34A-34C illustrate an example of generalization of spectral data based on sample interface effects according to some aspects.

FIG. 35 is a graph illustrating an example of high-reflectance background reference materials variations according to some aspects.

FIGS. 36A-36C illustrate the effect of changing the OPD on the spectra of a wavelength reference material according to some aspects.

FIGS. 37A and 37B illustrate examples of calibration transfer permutation trees according to some aspects.

FIG. 38 is an example of a process flow for optimizing the spectral device characteristics according to some aspects.

FIG. 39 illustrates an example of alteration of the distribution of the number of spectra versus the value of the analyte in the sample according to some aspects.

FIG. 40 is a process flow illustrating an example of optimization of the chemometrics model according to some aspects.

FIG. 41 is a diagram illustrating an example execution of the process shown in FIG. 40.

FIG. 42 is a diagram illustrating an example of a neural network according to some aspects.

FIG. 43 illustrates an example of optimizing the chemometrics model by applying wavelength selection according to some aspects.

FIG. 44 is a diagram illustraing an example of a spectral modeling adjustment process for applying model adjustement to further generalize the chemometrics model according to some aspects.

FIGS. 45A-45C illustrate adjustment of the chemometrics model to account for deviant spectral devices according to some aspects.

FIG. 46 is a diagram illustrating an example of unifying the spectral data coming from various spectral devices according to some aspects.

FIG. 47 is a diagram illustrating another example of unifying the spectral data coming from various spectral devices according to some aspects.

FIG. 48 is a process flow illustrating an example process for testing spectral devices according to some aspects.

FIG. 49 is a diagram illustrating an example of a globalization process for transferring a chemometrics model to a child spectral device corresponding to a different type of spectral device according to some aspects.

FIG. 50 is a diagram illustrating an example of testing child spectral devices according to some aspects.

FIG. 51 is a diagram illustrating a cloud-based spectral modeling system according to some aspects.

FIGS. 52A-52C are diagrams illustrating examples of generalized chemometrics model building using a generalizer spectral converter according to some aspects.

FIG. 53 is a diagram illustrating another example of a spectral modeling system according to some aspects.

FIG. 54 is a block diagram illustrating an example of a hardware implementation for a computing device employing a processing system according to some aspects.

FIG. 55 is a flow chart illustrating an exemplary process for producing a generalized chemometrics model according to some aspects.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Various aspects of the disclosure relate to techniques for building chemometrics (calibration) models for spectral devices targeting ultra-wide-scale deployment. In an aspect, a plurality of samples are measured on a subset of a plurality of spectral devices to generate corresponding spectral data. The subset may include a single spectral device or several spectral devices, but less than all of the plurality of spectral devices. In some examples, the spectral data may include measurements of phantom samples corresponding to the plurality of samples. The phantom samples may be formed of a stable substance having a same absorbance spectra as the plurality of samples.

In addition, a characteristics extractor generates a set of spectral device characteristics representing spectral variations in the plurality of spectral devices. The extracted spectral device characteristics may include, for example, one or more of signal-to-noise ratio (SNR), wavelength repeatability, wavelength error, absorbance scaling, self-apodization function, baseline shift, back reflection, thermal drift, environmental drift, optical path difference (OPD) variation, Etalon effect, or other suitable characteristic. The characteristics may be generated in various manners. For example, the characteristics extractor may include a plurality of stages for extracting the different spectral device characteristics. The stages may include, for example, background measurements of the plurality of spectral devices, reference material measurements of the plurality of spectral devices, narrowband emission measurements of the plurality of spectral devices, and temperature-control measurements of the plurality of spectral devices.

In some examples, the spectral device characteristics may be extracted by measuring universal samples on at least a portion of the plurality of spectral devices that are different than the samples used to obtain the spectral data on the subset of spectral devices. In some examples, the portion of the plurality of spectral devices may include all of the plurality of spectral devices. In other examples, the portion of the spectral devices may include selected spectral devices having corresponding spectral device characteristics covering a space of variations including corners of production line characteristics of a production line including the plurality of spectral devices. The measured spectra of the universal samples may be fed to a signal processor in the characteristics extractor to extract the spectral device characteristics of the plurality of spectral devices.

In other examples, instead of measuring universal samples, the spectral device characteristics may be generated based on statistical information related to the production line of the plurality of spectral devices. For example, the characteristics extractor may extract the spectral device characteristics based on an understanding of production line variations and histograms. In an example, the characteristics extractor may derive various statistical parameters, such as the mean value, standard deviation, skewness, or kurtosis, based on the statistical information and determine a probability distribution of each of the statistical parameters. The characteristics extractor may then generate the spectral device characteristics based on the statistical parameters and the respective probability distribution of each of the statistical parameters.

The spectral device characteristics generated by the characteristics extractor and the spectral data produced by the subset of the plurality of spectral devices may then be fed into a spectral converter to produce a plurality of artificial spectra representing remaining spectral devices of the plurality of spectral devices (e.g., the spectral devices not included in the subset). The artificial spectra represent the expected spectra to be generated on the remaining spectral devices based on the spectral device characteristics thereof. In some examples, the spectral converter further applies pre-processing to the spectral data produced by the subset of the plurality of spectral devices. For example, the spectral converter may apply a spectral variance function, a spectral correction function, a spectral modulation and perturbation function, or an optical head variance function to account for different variances in the spectral data.

The resulting artificial spectra, together with the original spectral data, may then be input to an artificial intelligence (AI) engine (also referred to herein as a chemometrics engine) to build a chemometrics model for one or more parameters associated with the samples. In some examples, the AI engine may be a cloud-based AI engine. The AI engine may then adjust the chemometrics model to account for deviant spectral devices that deviate in performance from other regular spectral devices. In addition, the AI engine may further generalize the chemometrics model to be appliable to different types of spectral device(s) using a transfer function generated based on measurements obtained from one or more of the plurality of spectral devices and the different types of spectral device(s). For example, the different types of spectral device(s) may use a different light modulator, optical head, or other spectral device configuration. In some examples, the chemometrics model may be used to calibrate one or more of the plurality of spectral devices. In addition, the chemometrics model may be used to characterize a sample under test measured on a test spectral device. In this example, the spectral device characteristics of the test spectral device, along with a sample measurement of the sample under test, may be fed to the AI engine to generate a result (e.g., measured value of the sample under test) using the chemometrics model.

Conventionally, the number of bench-top infrared spectrometers produced per year was limited due to the expense of such bulky devices. With the technological advancement of a new generation of spectrometers and new usage models of the spectrometers, the number of devices being produced and sold is significantly growing. The new generation makes use of different technologies and methods to produce miniaturized spectrometers. For example, the new generation includes diffraction-grating spectrometers using a digital micro-mirror device (DMD) together with a diffraction grating, or a scanning micro-electro-mechanical-systems (MEMS) diffraction grating rotated by a rotary MEMS actuator with a spectral range of 950 nm to 1.9 μm. The new generation further includes MEMS tunable Fabry-Perot devices covering wavelength ranges 1.5-2.0 μm or 1.9-2.5 μm, and MEMS Fourier Transform Infrared (FTIR) spectrometers based on self-aligned and highly integrated architectures, such as Michelson interferometers, multimode interference MMI, or spatially-shifted Fabry-Perot. In addition, the new generation further includes hand-held spectral sensing devices using any of the above core spectral sensor designs.

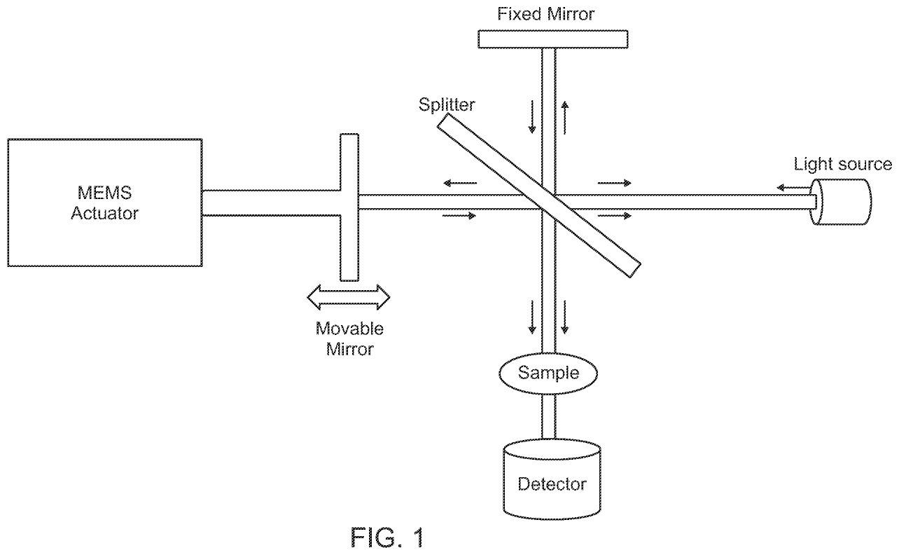

FIG. 1 is a diagram illustrating an example of a spectrometer 100 according to some aspects. The spectrometer 100 may be, for example, a Fourier Transform infrared (FTIR) spectrometer that exploits light interference and Fourier transform to calculate the spectral content of an infrared light beam. In the example shown in FIG. 1, the spectrometer 100 is a Michelson FTIR interferometer.

FTIR spectrometers measure a single-beam spectrum (power spectral density (PSD)), where the intensity of the single-beam spectrum is proportional to the power of the radiation reaching the detector. In order to measure the absorbance of a sample 112, the background spectrum (i.e., the single-beam spectrum in absence of a sample) may first be measured to compensate for the instrument transfer function. The single-beam spectrum of light transmitted or reflected from the sample 112 may then be measured. The absorbance of the sample 112 may be calculated from the transmittance, reflectance, or trans-reflectance of the sample 112, the former being illustrated. For example, the absorbance of the sample 112 may be calculated as the ratio of the spectrum of transmitted light, reflected light, or trans-reflected light from the sample to the background spectrum.

The FT-IR spectrometer 100 includes a fixed mirror 106, a moveable mirror 108, a beam splitter 104, and a detector 114 (e.g., a photodetector). A light source 102 associated with the spectrometer 100 is configured to emit an input beam and to direct the input beam towards the beam splitter 104. The light source 102 may include, for example, a laser source, one or more wideband thermal radiation sources, or a quantum source with an array of light emitting devices that cover the wavelength range of interest.

The beam splitter 104 is configured to split the input beam into two beams. One beam is reflected off of the fixed mirror 106 back towards the beam splitter 104, while the other beam is reflected off of the moveable mirror 108 back towards the beam splitter 104. The moveable mirror 108 may be coupled to an actuator 110 to displace the movable mirror 108 to the desired position for reflection of the beam. An optical path length difference (OPD) is then created between the reflected beams that is substantially equal to twice the mirror 108 displacement. In some examples, the actuator 110 may include a micro-electro-mechanical systems (MEMS) actuator, a thermal actuator, or other type of actuator.

The reflected beams interfere at the beam splitter 104 to produce an output light beam, allowing the temporal coherence of the light to be measured at each different Optical Path Difference (OPD) offered by the moveable mirror 108. The signal corresponding to the output light beam may be detected and measured by the detector 114 at many discrete positions of the moveable mirror 108 to produce an interferogram. In some examples, the detector 114 may include a detector array or a single pixel detector. The interferogram data verses the OPD may then be input to a processor (not shown, for simplicity). The spectrum may then be retrieved, for example, using a Fourier transform carried out by the processor.

In some examples, the spectrometer 100 may be implemented as a MEMS interferometer (e.g., a MEMS chip). For example, the MEMS chip may be attached to a printed circuit board (PCB) that may include, for example, one or more processors, memory devices, buses, and/or other components. As used herein, the term MEMS refers to an actuator, a sensor, or the integration of sensors, actuators and electronics on a common silicon substrate through microfabrication technology to build a functional system. Microelectronics are typically fabricated using an integrated circuit (IC) process, while the micromechanical components are fabricated using compatible micromachining processes that selectively etch away parts of the silicon wafer or add new structural layers to form the mechanical and electromechanical components. One example of a MEMS element is a micro-optical component having a dielectric or metallized surface working in a reflection or refraction mode. Other examples of MEMS elements include actuators, detector grooves and fiber grooves.

In some examples, the MEMS interferometer (FT-IR spectrometer 100) may be fabricated using a Deep Reactive Ion Etching (DRIE) process on a Silicon On Insulator (SOI) wafer in order to produce the micro-optical components and other MEMS elements that are able to process free-space optical beams propagating parallel to the SOI substrate. For example, the electro-mechanical designs may be printed on masks and the masks may be used to pattern the design over the silicon or SOI wafer by photolithography. The patterns may then be etched (e.g., by DRIE) using batch processes, and the resulting chips (e.g., MEMS chip) may be diced and packaged (e.g., attached to the PCB).

In some examples, the beam splitter 104 may be a silicon/air interface beam splitter (e.g., a half-plane beam splitter) positioned at an angle (e.g., 45 degrees) from the input beam. The input beam may then be split into two beams L1 and L2, where L1 propagates in air towards the moveable mirror 108 and L2 propagates in silicon towards the fixed mirror 106. Here, L1 originates from the partial reflection of the input beam from the half-plane beam splitter 104, and thus has a reflection angle equal to the beam incidence angle. L2 originates from the partial transmission of the input beam through the half-plane beam splitter 104 and propagates in silicon at an angle determined by Snell's Law. In some examples, the fixed and moveable mirrors 106 and 108 are metallic mirrors, where selective metallization (e.g., using a shadow mask during a metallization step) is used to protect the beam splitter 104. In other examples, the mirrors 106 and 108 are vertical Bragg mirrors that can be realized using, for example, DRIE.

In some examples, the MEMS actuator 110 may be an electrostatic actuator formed of a comb drive and spring. For example, by applying a voltage to the comb drive, a potential difference results across the actuator 110, which induces a capacitance therein, causing a driving force to be generated as well as a restoring force from the spring, thereby causing a displacement of moveable mirror 108 to the desired position for reflection of the beam back towards the beam splitter 104.

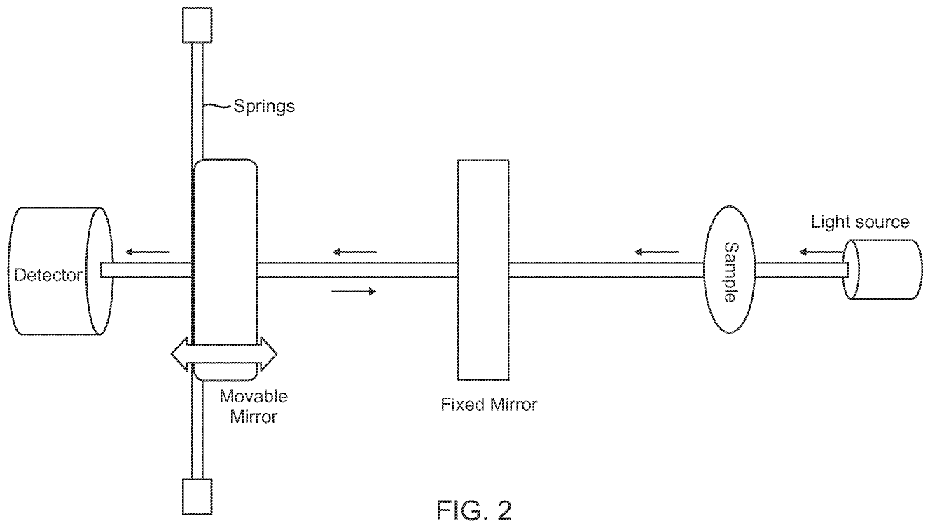

FIG. 2 is a diagram illustrating another example of a spectrometer 200 according to some aspects. The spectrometer 200 may be, for example, a Fabry-Perot spectrometer that includes a fixed mirror 206, a moveable mirror 208, and a detector 212 (e.g., a photodetector). A light source 202 associated with the spectrometer 200 is configured to emit an input beam and to direct the input beam towards the fixed mirror 206. The light source 202 may include, for example, a laser source, one or more wideband thermal radiation sources, or a quantum source with an array of light emitting devices that cover the wavelength range of interest.

For light trapped in a Fabry—Perot cavity formed between the fixed mirror 206 and the movable mirror 208, maximum transmission occurs when the optical path difference between each transmitted beam is equal to one complete cycle. This phenomenon can be used to create a tuneable light filter, which can be used as a spectrometer, as shown in FIG. 2. According to the phase shift equation, the wavelength of maximum transmission is given by:

λₘₐₓ=2nᵣd*cos(θ) (1)

where nᵣ is the refractive index of the cavity, d is the distance between the two mirrors 206 and 208 and θ is the incidence angle. If d changes as a result of motion of the movable mirror 208 using, for example, springs 210, λₘₐₓ will change, thus forming a spectrometer. By measuring the light intensity using the photodetector 212 and measuring d, the relative intensity of each wavelength can be calculated.

As with the FT-IR spectrometer 100 shown in FIG. 1, a single-beam spectrum (power spectral density (PSD)) may be obtained using the Fabry-Perot spectrometer 200 shown in FIG. 2, where the intensity of the single-beam spectrum is proportional to the power of the radiation reaching the detector 212. In order to measure the absorbance of a sample 204, the background spectrum (i.e., the single-beam spectrum in absence of a sample) may first be measured to compensate for the instrument transfer function. The single-beam spectrum of light transmitted or reflected from the sample 204, the former being illustrated, may then be measured.

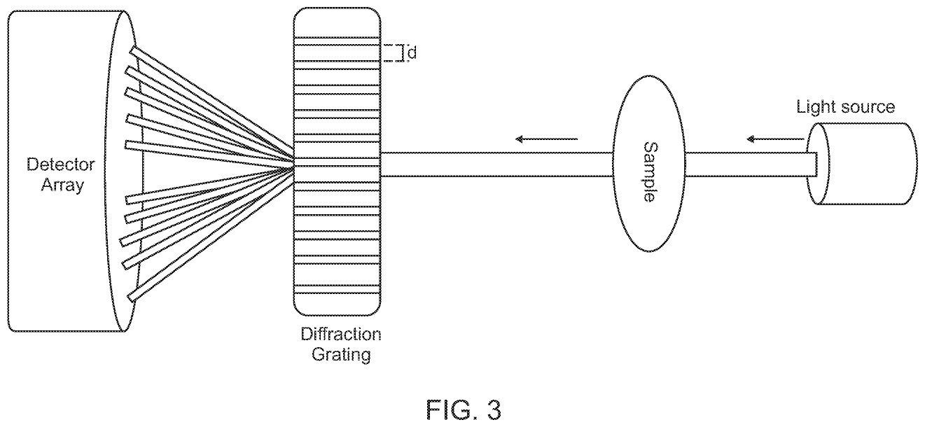

FIG. 3 is a diagram illustrating another example of a spectrometer 300 according to some aspects. The spectrometer 300 may be, for example, a diffraction grating spectrometer that includes a diffraction grating 306 and a detector 308 (e.g., a photodetector). A light source 302 associated with the spectrometer 300 is configured to emit an input beam and to direct the input beam towards the diffraction grating 306. The light source 302 may include, for example, a laser source, one or more wideband thermal radiation sources, or a quantum source with an array of light emitting devices that cover the wavelength range of interest.

Diffraction grating spectrometers 300, such as the one shown in FIG. 3, exploit the diffraction to analyze light content at different angles, according to the equation

d sin(θₘ)=mλ (2)

where d is the periodicity of the grating, θₘ is the angle of diffracted beam and m is the order of diffraction. By measuring light intensity at each position on the detector 308, relative intensity for each wavelength point can be calculated. In some examples, the detector 308 may be a multi-pixel detector, as shown in FIG. 3, to detect the different intensity of light on every point on the detector 308 and convert that to an image that can then be processed to produce the light spectrum. In other examples, a single detector can be used. However, a movable mirror or slit may be needed to direct each wavelength separately to the detector.

The resulting light spectrum produced by the diffraction grating spectrometer 300 corresponds to a power spectral density (PSD), where the intensity of the spectrum is proportional to the power of the radiation reaching the detector 308 at each point. In order to measure the absorbance of a sample 304, the background spectrum (i.e., the spectrum in absence of a sample) may first be measured to compensate for the instrument transfer function. The spectrum of light transmitted or reflected from the sample 304, the former being illustrated, may then be measured.

The unique information from the vibrational absorption bands of a molecule are reflected in an infrared spectrum that may be produced, for example, by any of the spectrometer shown in FIGS. 1-3. By applying spectral numerical processing and statistical analysis to a spectrum, the information in the spectrum may be identified or otherwise classified. The application of statistical methods to the analysis of experimental data is traditionally known as chemometrics, and more recently as artificial intelligence.

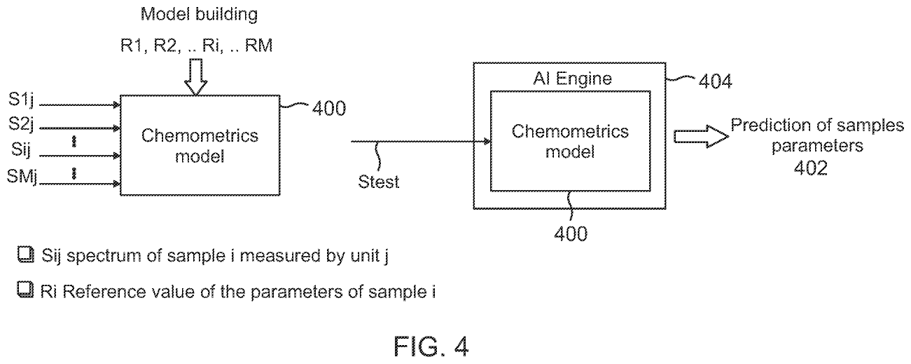

FIG. 4 is a diagram illustrating an example of a chemometrics model 400 according to some aspects. The chemometrics model 400 (also referred to herein as a calibration model) shown in FIG. 4 may be built for a certain device (denoted as device j or unit j) by measuring samples spanning the range of variation in the analyzed parameters. For example, M samples (numbered from 1 to i to M) are measured each with a spectrum Sij produced by the device j. Reference values for the different parameters of the sample i are denoted by the vector Ri, where the vector length is the number of parameters analyzed per sample. For example, the samples can also be measured by conventional methods and the values of various parameters associated with the samples may be recorded as reference values. Given the reference values R1, R2, . . . Ri, . . . RM corresponding to the spectra S1j, S2j, . . . Sij, . . . SMj, the chemometrics model 400 can be built and the coefficients of the multivariate regression can be found. Other types of models that are non-linear in nature, such as neural networks, may be used as well.

The resulting chemometrics model 400 may then be applied to a spectrum Sₜₑₛₜ of a sample under test to produce a result 402 (e.g., a prediction of a parameter) associated with the sample. In some examples, the chemometrics model 400 is included within an AI engine 404 and the spectrum may be input to the AI engine 404 for analysis and processing. The AI engine 404 is configured to process the spectrum to generate a result 402 indicative of at least one parameter associated with the sample from the spectrum. For example, the AI engine 404 may include one or more processors for processing the spectrum and a memory configured to store one or more calibration (chemometrics) models utilized by the processor in processing the spectrum. The AI engine 404 can include, for example, one or more calibration models, each built for a respective type of analyte under test. Validation and outliers detection of the test results may then be performed to refine the chemometrics model 400.

In some examples, the spectrum includes a measured absorption spectra and the AI engine 404 is configured to detect one or more analytes from absorption signals of the measured absorption spectra in the near-infrared frequency range. In some examples, absorption signals in the near-infrared region (frequency range) can be used to detect the analyte based on overtones and combinations of the fundamental vibrational modes. Since the spectrum produced by infrared (IR) spectroscopy are instantaneous, unlike conventional analysis methods, there is no need to wait for certain transformations (e.g., chemical transformations) to occur within the sample. Different physical and chemical parameters of the sample can be analyzed with a single scan.

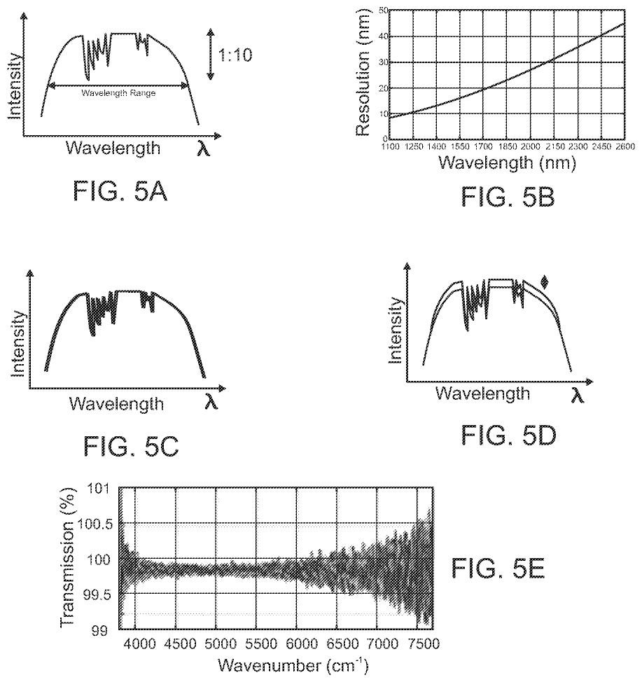

Various specifications (characteristics) of spectral sensing devices may affect the obtained spectrum when measuring a sample under test. The spectral range defines the minimum and maximum wavelength. In an FTIR spectrometer, the spectral range is limited by the detector responsivity and the beam splitter material transparency. The range is usually defined based on a certain drop in the signal level, such as, for example, a ratio of 1 to 10 as shown in FIG. 5A. The spectral resolution in an FTIR spectrometer is inversely proportional with the mirror travel range, such that the wavenumber resolution Δν˜1/xₘₐₓ, where xₘₐₓ is the maximum travel range representing the FT window width. The resolution in the wavelength domain increases with the wavelength following the relation Δλ=Δνλ². An example is shown in FIG. 5B for Δν=66 cm⁻¹. The wavelength accuracy indicates how much the spectrum is x-axis shifted from the true value of the spectrum, as shown in FIG. 5D. This may further be affected by the accuracy of the mirror position. The photometric accuracy indicates how much the absorbance spectrum is y-axis shifted from the true value, as shown in FIG. 5D. The signal-to-noise ratio (SNR) is the strength of the signal carrying information to that of unwanted interference. In FTIR spectrometers, the SNR is usually extracted using a 100% line method, in which several measurements (typically 50-100) are carried out consecutively and each measurement is normalized to the preceding one, as shown in FIG. 5E. The noise can then be extracted from the root mean square variation Nᵣₘₛ of the 100% line. The SNR is calculated as 100/Nᵣₘₛ.

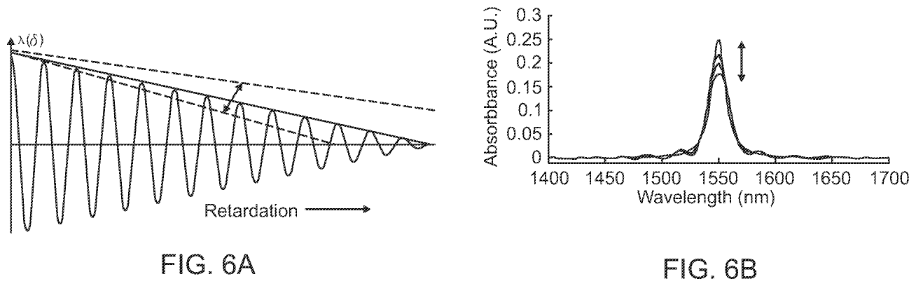

Due to statistical variations in the production line of the components of a spectral device and the system integration of these components, there is a difference in the specifications (also referred to herein as spectral device characteristics) of the different units (spectral devices) coming out of the same production line. These difference include x-axis values, y-axis values and exact shape and width of the spectral lines. One example is self-apodization that occurs in FTIR spectrometers seen as an attenuation in the interferogram of a single wavelength line versus optical path difference or the retardation, as shown in FIG. 6A. This depends on the divergence angle of the input light beam and on the alignment errors (for example inclination angles) of the surfaces of the interferometer. Variations in the self-apodization envelope, as shown in FIG. 6A, can lead to variations in the absorbance spectrum, as shown in in FIG. 6B. Different effects can also arise from variations related to the photodetector response, light source, optical coupling elements between the light source, interferometer and photodetector, background samples, distance between the sample and the optical windows, actuation and sensing electronics.

These variations can lead to errors in the resolution of the line as well as the photometric value of the absorbance. Due to these variations and errors, the chemometrics model developed on a certain spectral device may not be usable with acceptable prediction errors on another spectral device. This phenomenon can be seen as a systematic bias and/or proportional bias between spectral devices (units), with systematic bias being the most common. The systematic bias (Bⱼ) is the mean difference between predicted and reference methods (offset) given by (where j refers to a specific device):

$${B}_{j} = \frac{1}{M} {\sum }_{i = 1}^{M} Y p r e {d}_{j i} - Y r e {f}_{i} = \overline{{Y}_{pred J}} - \overline{{Y}_{ref}} \qquad ( 3 )$$

The standard error of prediction (SEPⱼ) is calculated as the standard deviation of predicted residuals:

$$S E {P}_{j} = \sqrt{\frac{1}{M - 1} {\sum }_{i = 1}^{M} {( Y p r e {d}_{i} - Y r e {f}_{i} - B )}^{2}} \qquad ( 4 )$$

The root mean squared error (RMSE) can then be calculated as:

$$R M S {E}_{j} = \sqrt{\frac{1}{M - 1} {\sum }_{i = 1}^{M} {( Y p r e {d}_{i} - Y r e {f}_{i} )}^{2}} \approx \sqrt{S E {P}^{2} + {B}^{2}} \qquad ( 5 )$$

Due to the unit-to-unit variation, large bias or/and slope errors in the predictions may occur. In this case, a calibration transfer may be used to transfer the developed calibration model to another spectral sensing device that may have a different architecture or different specifications. The transfer is carried out either by transferring the spectra or transferring the whole model predictions. In the first case, a set of reference samples can be measured on unit j (original device) and on unit N (targeted device). The resulting spectra can be used to find the mathematical relation for conversion of the spectra measured on unit j to the equivalent spectra that should result from being measured on unit N. Then, the normal chemometrics modeling is applied on the transferred spectra. Direct standardization (DS) and piecewise direct standardization (PDS) are the common methods that are used to transfer the spectra by finding a regression relation between the spectral points of the two devices. DS uses the whole slave spectrum while in PDS, a small window from the slave spectrum is used instead. In the second case, mathematical operations are applied to convert or correct the regression coefficient of the model of unit j so that it can provide correct results with unit N. Data processing may also be used, such as baseline removal, de-trending and derivatives, multiplicative scatter correction, orthogonal signal correction and generalized least squares. One approach, called spectral space transformation, has been developed to maintain the predictive abilities of multivariate calibration models when the spectrometer or measurement conditions are altered. This approach attempts to eliminate the spectral differences induced by the changes between devices or measurement conditions. Another approach based on 1-norm or 2-norm variants of Tikhonov regularization can also be used to perform calibration maintenance and transfer where just a few samples measured in the secondary condition/device are augmented to the primary calibration data to update the primary model. The main challenge for all the transfer methods is that in most of the cases, a material/device-specific matrix is generated in order to develop transfer models per each material/device.

For example, the use of NIR spectroscopy in the feed industry has historically been hindered by the need to build a calibration model for each spectral device. Therefore, calibration transfer appeared as a necessity for practical application and implementation of NIR over time. The spectral devices were benchtop devices used in the laboratories and produced with relatively small volume. The calibration transfer involved the use of tens of samples measured on the primary (or original) device and the secondary (or target) device for the transfer. The aforementioned different approaches attempt to simplify the mathematics and reduce the computational and experimental measurements burden. However, there is still a need to measure a subset of standardization samples on two devices or under two sets of experimental conditions. The standardization samples have to be of the same type as the samples being modeled and contain analytes spanning the same range of analyzed parameters. In addition, the samples have to be measured on each and every new spectral device the calibration (chemometrics) model is being transferred to. Moreover, intrinsic to the nature of the sample, the measurements on both devices should be performed at the same time to avoid unexpected changes in the standardization samples. Thus, a different chemometrics model has to be installed on each different spectral device produced. This is not compatible with the mass production of chemometrics models and the ultra-wide-scale deployment of the spectral devices for ubiquitous chemical analysis.

Based on the chemical nature of the problem that the intrinsic absorbance of the substances in a certain sample remains unchanged, if the optical path and spectral device response are the same, a simulation for the direct representation of the extended version of the Beer-Lambert law for multi-component systems can be used based on the equation:

A=b[ε₁(λ)c₁+ε₂(λ)c₂+ . . . εₙ(λ)cₙ] (6)

where A is the absorbance, ε is the substance absorptivity and c is the substance concentration in the sample. Thus, using experimental measurements for the absorptivity and solvent displacement values for each substance in the sample matrix, and by measuring the background of each individual instrument b, the absorbance and transmittance can be calculated. This method has been presented on liquid analysis problem. However, this method did not produce satisfactory results for the spectral device that has deviations in its spectral response. Additional measurements for reference samples by the different spectral devices had to be used to complement the simulation data. In addition, although the prediction of the spectra based on Equation 6 above can be satisfactory for liquids or gases since the physical nature of the sample (e.g., in terms of shape, path length, and scattering) are well controlled, for solids and inhomogeneous or heterogeneous samples, this may not be able to be represented by Equation 6.

A different approach is to measure a very large data set using many different spectral devices to build a global calibration model that can be used on all of them. However, pre-processing of the data set should be carefully done to reduce the variation between the spectral devices to reduce the global prediction errors. Moreover, measuring large datasets on large numbers of spectral devices can be very expensive and not practical in some cases.

Therefore, various aspects relate to techniques for building chemometrics (calibration) models for spectral devices targeting ultra-wide-scale deployment. In various examples, a calibration transfer from a first spectral device to a second spectral device may be carried out without measuring the same samples on both spectral devices, thus reducing the cost of the sample measurement process and enabling globalization of chemometric models. Instead, spectral device characteristics of the first and second spectral devices may be generated by a characteristics extractor and the output thereof fed to a spectral converter to produce artificial spectra for the second spectral device based on spectral data obtained by the first spectral device. The artificial spectra and spectral data may then be fed into to a chemometrics modeler (e.g., AI engine) to produce the chemometrics model for the second spectral device. The chemometrics model may further be generalized for a plurality of spectral devices based on the respective spectral device characteristics thereof.

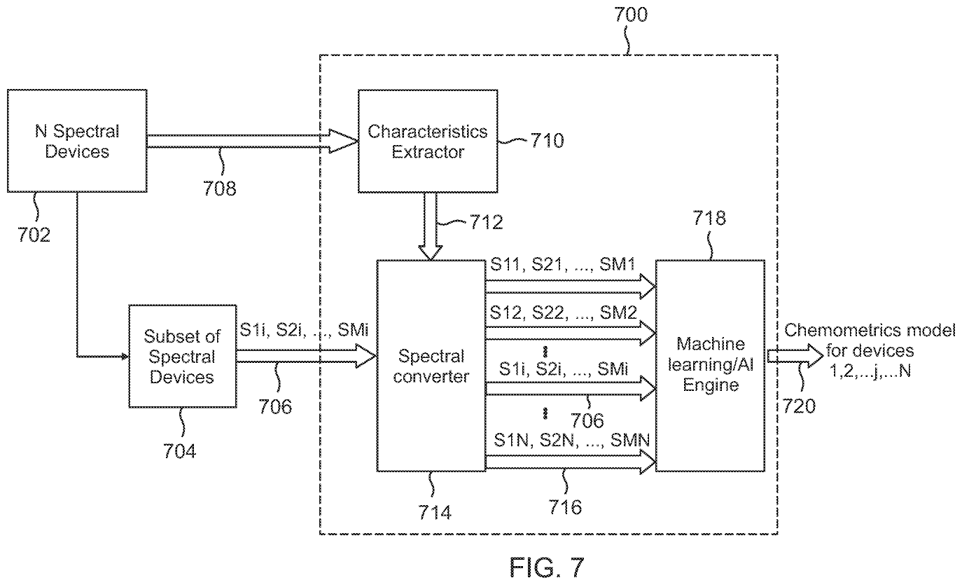

FIG. 7 is a diagram illustrating an example of a spectral modeling system 700 according to some aspects. The spectral modeling system 700 includes a characteristics extractor 710, a spectral converter 714, and a machine learning/AI engine 718. The characteristics extractor 710 is configured to receive spectral device information 708 related to a plurality of spectral devices 702 (e.g., N spectral devices). Each spectral device 702 may correspond, for example, to one of the spectral devices shown in FIG. 1, 2, or 3. In some examples, the N spectral devices 702 represent a production line of spectral devices having a same or similar configuration. The spectral device information 708 may indicate specification (characteristic) variations between the plurality of spectral devices 702. In some examples, the spectral device information 708 may include measured spectra of background or reference material or measured spectra of universal samples from at least a portion of the spectral devices 702. In other examples, the spectral device information 708 may include other spectral device information, such as statistical information related to the production line of spectral devices 702.

The characteristics extractor 710 is configured to generate spectral device characteristics 712 representing spectral variations in the plurality of spectral devices 702 based on the spectral device information 708 and to input the spectral device characteristics 712 to the spectral converter 714. In some examples, the spectral device characteristics 712 may include one or more of signal-to-noise ratio (SNR), wavelength repeatability, wavelength error, absorbance scaling, self-apodization function, baseline shift, back reflection, temperature variation (e.g., thermal drift), environmental drift, optical path difference (OPD) variations, or Etalon effect.

A subset of the spectral devices 704 (e.g., i spectral devices, where i is <<N) may be used to measure a plurality of samples (e.g., M samples). The resulting spectral data 706 of the measured samples may be fed into the spectral converter 714. The spectral data 706 may include a respective spectrum S1i, S2i, SMi from each of the i spectral devices 704 for each of the M samples. The spectral converter 714 may apply multiple mathematical transformations and spectral effects to the spectral data 706 using the spectral device characteristics 712 to generate a plurality of artificial spectra 716 representing remaining spectral devices (e.g., spectral devices not included in the subset 704) the plurality of spectral devices 702. For example, as shown in FIG. 7, the resulting artificial spectra may include S11, S21, . . . , SM1 for spectral device 1, S12, S22, . . . , SM2 for spectral device 2, and so on through S1N, S2N, . . . , SMN for spectral device N. The artificial spectra 716 should resemble the spectra expected to be produced by the corresponding spectral device 702 (e.g., with certain spectral device characteristics).

The artificial spectra 716, along with the spectral data 706 (e.g., S1i, S2i, . . . , SMi) obtained by the subset of spectral devices, may be fed into the AI engine 718 to produce a respective chemometrics model 720 for one or more parameters associated with the samples. Although not shown in FIG. 7, it should be understood that the chemometrics models 720 may be built using reference values for different parameters of the plurality of samples. The chemometrics model may then be used by the plurality of spectral devices in predicting sample parameters based on actual measured samples, as shown in FIG. 4.

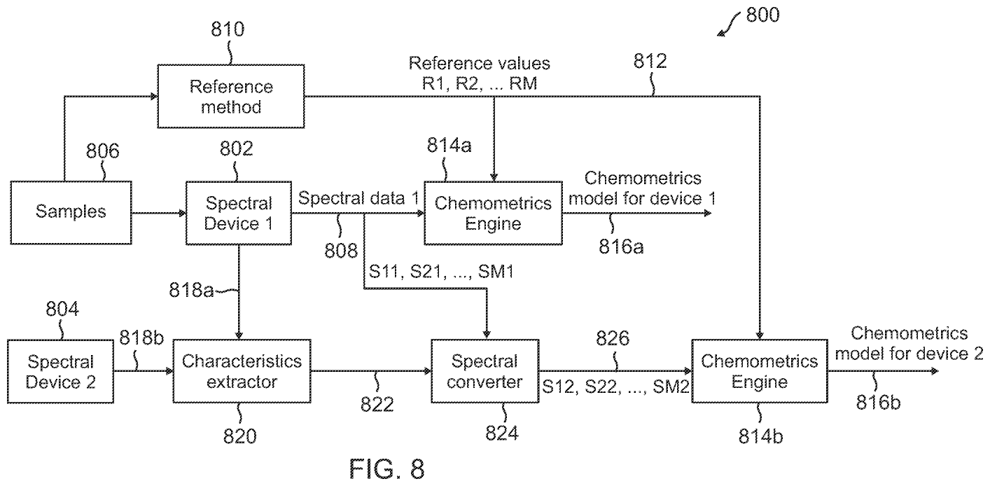

FIG. 8 is a diagram illustrating an example of a spectral modeling system 800 for two spectral devices 802 and 804 according to some aspects. In the example shown in FIG. 8, a first spectral device (Device 1) 802 is configured to measure a plurality of samples (e.g., M samples) 806 and to produced spectral data 808 (e.g., S11, S21, . . . , SM1) representative of the plurality of samples. The spectral data 808 together with references values 812 (e.g., R1, R2, . . . , RM) of analyzed parameters in the samples 806, obtained by a reference method 810, are fed to a chemometrics engine 814a (e.g., AI engine) to build a chemometrics model 816a for Device 1 802.

To produce a chemometrics model 816b for a second spectral device (Device 2) 804, a chemometrics (calibration) transfer may be performed without using any of the M samples 806. A characteristics extractor 818 (which may correspond, for example, to the characteristics extractor 710 shown in FIG. 7) may be configured to generate spectral device characteristics 822 representing spectral variations in Device 1 802 and Device 2 804 based on spectral device information 818a and 818b associated with each of Device 1 802 and Device 2 804. In some examples, the spectral device information 818a and 818b may include measured spectra of background or reference material or measured spectra of universal samples different than the samples 806. In other examples, the spectral device information 818a and 818b may include other spectral device information, such as statistical information related to the spectral devices 802 and 804. In some examples, the spectral device characteristics 822 may include one or more of signal-to-noise ratio (SNR), wavelength repeatability, wavelength error, self-apodization function, baseline shift, back reflection, or temperature variation (e.g., thermal drift).

The spectral device characteristics 822, together with the spectral data 808 may then be fed into a spectral converter 824. The spectral converter 824 can then produce generated (artificial) spectra 826 for Device 2 804 resembling the spectra expected to be produced by Device 2 804 if Device 2 804 had measured the samples 806. The artificial spectra 826 may then be fed to a chemometrics engine 814b to produce the chemometrics model 816b for Device 2 804. In some examples, the chemometrics engines 814a and 814b may be combined into a single chemometrics engine.

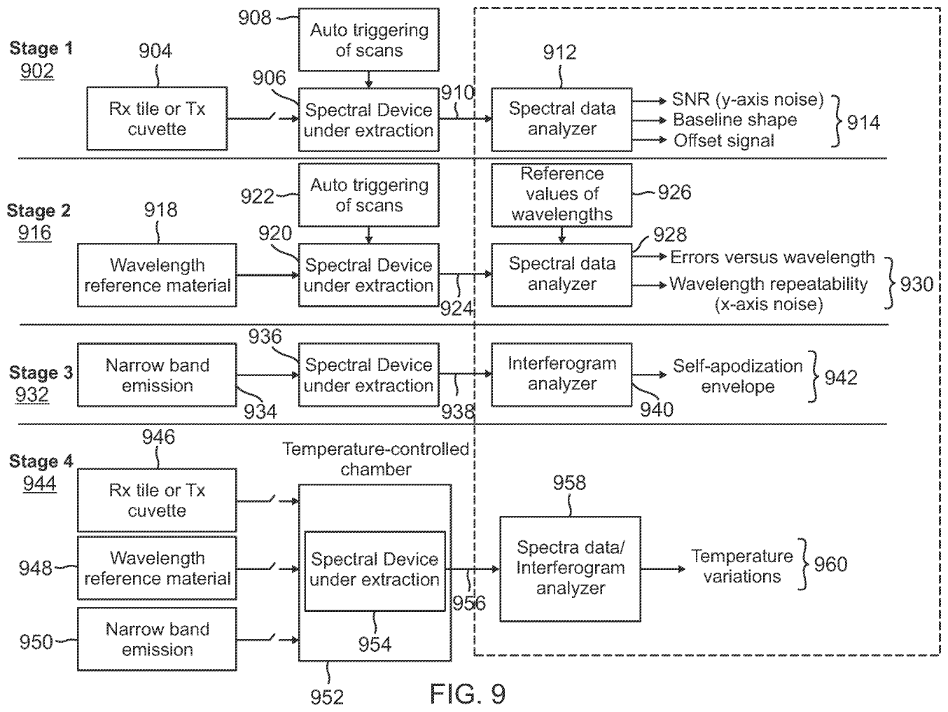

FIG. 9 is a diagram illustrating an example of a characteristics extractor 900 according to some aspects. In the examples shown in FIG. 9, the characteristics extractor 900 includes a plurality of stages (Stage 1 902, Stage 2 916, Stage 3 932, and Stage 4 944). However, it should be understood that the characteristics extractor 900 may include a combination of one or more of the shown stages. In some examples, the characteristics extractor 900 may be configured to produce a plurality of spectral device characteristics, such as the characteristics shown in Table 1 below.

TABLE 1

| Characteristic | Description |
|---|---|
| SNR | Random values on y-axis with |
| | wavelength dependence |
| Wavelength | Random values on x-axis with |
| repeatability | wavelength dependence |
| Wavelength error | Shift in x-axis with zero, first, or higher |
| | order dependence on wavelength |
| Self-apodization | Attenuation of the single wavelength |
| | interferogram with optical path difference. |
| | The attenuation is a function of |
| | wavelength. |
| Baseline shift | Shift in y-axis with zero, first, or higher |
| | order dependence on wavelength |
| Back reflection/ | Offset and scaling in y-axis with zero, |
| offset signal | first, or high order dependence on |
| | wavelength |
| Temperature | Variation in light modulation components |
| variations | or photodetector wavelength response |
| (thermal drift) | verses temperature |

Additional characteristics may include, for example, absorbance scaling, environmental drift, optical path difference (OPD) variations, Etalon effects, and other suitable spectral device characteristics.

In Stage 1 902, a background spectra 910 is extracted by a spectral device 906 using a reflection tile or transmission sampling accessory (e.g., a cuvette) 904. Using auto triggering of scans 908, several measurements can be taken and fed into a spectral data analyzer 912 of the characteristics extractor 900 to extract the SNR 914 by calculating the changes occurring in the captured response from one measurement to another representing the noise on the y-axis. The baseline shape can be also extracted from the spectral data. When the scan is carried out without having the tile or the cuvette 904 in place, the captured signal is an offset signal in the spectrum that can be coming from internal stray light in the spectral device 906 not passing by the sample.

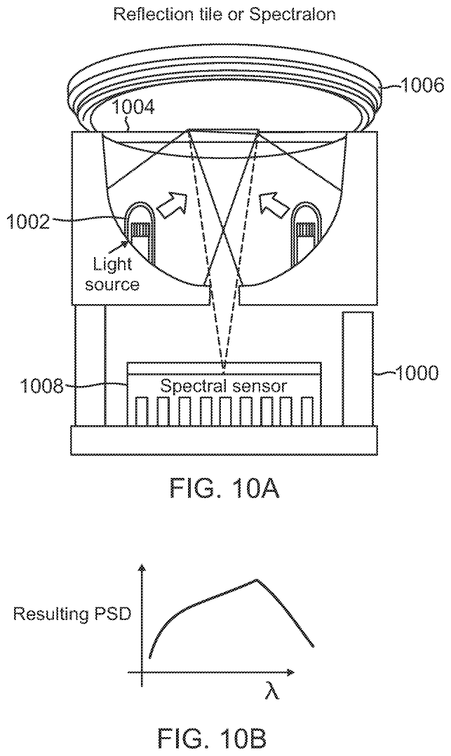

FIG. 10A is a diagram illustrating an example of a spectral device 1000 configured for Stage 1 characteristic extraction according to some aspects. As can be seen in the example shown in FIG. 10A, the spectral device 1000 includes one or more light sources 1002 arranged to direct incident light towards a transparent window 1004 on a top surface of the spectral device 1000. A reflection tile or spectralon 1006 may be positioned on the transparent window 1004 to reflect light towards a spectral sensor configured to produce a single-beam spectrum (power spectral density (PSD)), as shown in FIG. 10B. The resulting PSD is indicative of the SNR of the spectral device 1000.

Referring again to FIG. 9, in Stage 2 916, a wavelength reference material 918 is used to capture measured spectra 924 by a spectral device 920. The material 918 has multi-peaks in its spectral response and the location and amplitudes of these peaks are stable versus time. The material can be, for example, polystyrene, Talc powder, and/or a mixture of rare-earth oxides. Using auto triggering of scans 922, several measurements can be taken and fed into a spectral data analyzer 928 of the characteristics extractor 900 to extract the wavelength repeatability 930. For example, with auto triggering of scans 922, such that the spectra 924 are captured several times, the repeatability 930 of the locations of the peaks can be obtained representing the noise on the x-axis of the spectral data. In addition, by comparing the locations of the peaks with respect to reference values 926, the wavelength errors 930 can be obtained. The reference values 926 can be obtained by measuring the reference material 918 on a reference device (not shown) that has the same resolution and apodization of the spectral device 920 under extraction. In other examples, the reference spectrum 926 can be obtained from a device that has much finer resolution. In this example, the reference spectrum 926 can be convoluted with the line shape function corresponding to the resolution and apodization target of the spectral device 920 under extraction.

In Stage 3 932, a narrowband optical filter 934 (e.g., a filter receiving wideband light and passing narrowband light) may be measured by a spectral device 936, and the resulting interferogram 938 may be fed into an interferogram analyzer 940 of the characteristics extractor 900 to extract a self-apodization function (envelope) of the spectral device 936. In this case, the interferogram 938 corresponds to measured light with a narrow band. The spectral width of the light source should be much smaller than the resolution of the spectral device 936 under extraction. Otherwise, a correction can be applied to account for the effect of the larger spectral width of the light source.

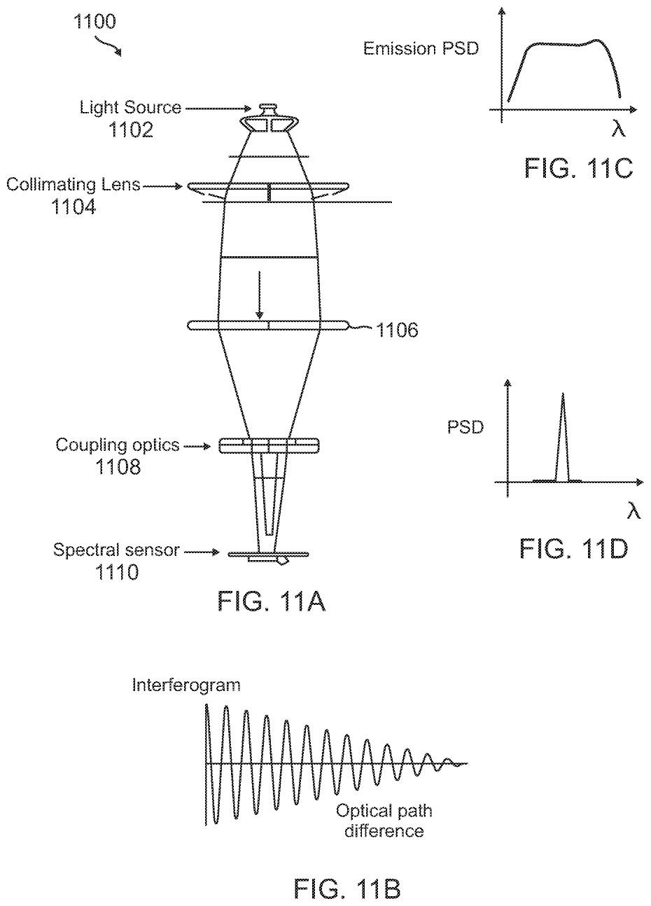

FIG. 11A is a diagram illustrating an example of a spectral device 1100 configured for Stage 2 or Stage 3 characteristic extraction according to some aspects. The spectral device 1100 includes a light source 1102, a collimating lens 1104, a reference material for Stage 2 or optical filter 1106 for Stage 3, coupling optics 1108 and a spectral sensor 1110. Incident light from the light source 1102 may be directed via the collimating lens 1104 to the reference material/optical filter 1106 in a transmission mode. Output light passing through the reference material/optical filter 1106 may be coupled via the coupling optics to the spectral sensor 1110 for generation of an interferogram, as shown in FIG. 11B. The light source 1102 may have an emission power spectral density (PSD) as shown in FIG. 11C. In addition, for Stage 3, the optical filter 1106 may have a narrowband emission, as shown in FIG. 11D.

Referring again to FIG. 9, in Stage 4 944, the variations in the spectral response due to the temperature variations are obtained. In this case, the spectral device 954 under extraction is inserted into a temperature-controlled chamber 952. The temperature of the chamber 952 is varied and one or more materials are measured, such as the reflection tile or the transmission cuvette 946, the wavelength reference material 948, and/or the narrow band optical filter 950. In this example, the resulting spectrum 956 may be fed into a spectral data/interferogram analyzer 958 of the characteristics extractor 900 to obtain the matrix coefficients between the extracted spectral characteristics and the temperature indicative of the temperature variations (e.g., thermal drift) 960.

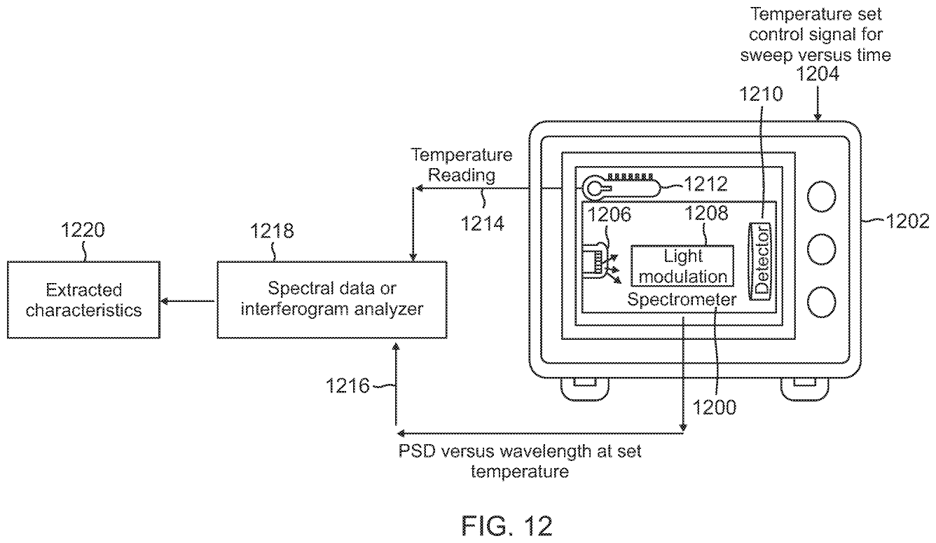

FIG. 12 is a diagram illustrating an example extractor setup for Stage 4 according to some aspects. In the example shown in FIG. 12, a spectral device 1200 may be inserted into a temperature-controlled chamber 1202. The temperature of the temperature-controlled chamber 1202 may be controlled via a control signal 1204 that can be configured to sweep the temperature verses time. The spectral device 1200 may include, for example, a light source 1206 and a spectral sensor including a light modulator 1208 and a detector 1210. A thermometer 1212 may be included in the temperature-controlled chamber 1202 to output a temperature reading 1214 of the temperature-controlled chamber 1202. The temperature reading 1214 and spectrum/interferogram 1216 (e.g., PSD verses wavelength at the set temperature 1214) may be input to a spectral data/interferogram analyzer 1218 for extraction of one or more characteristics 1220 (e.g., thermal drift) of the spectral device 1200.

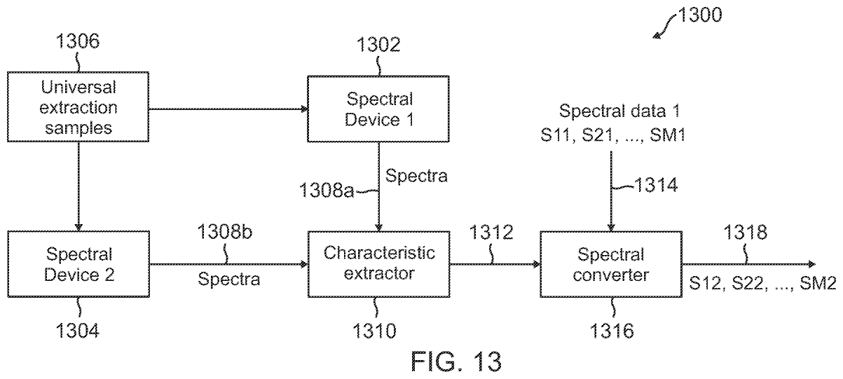

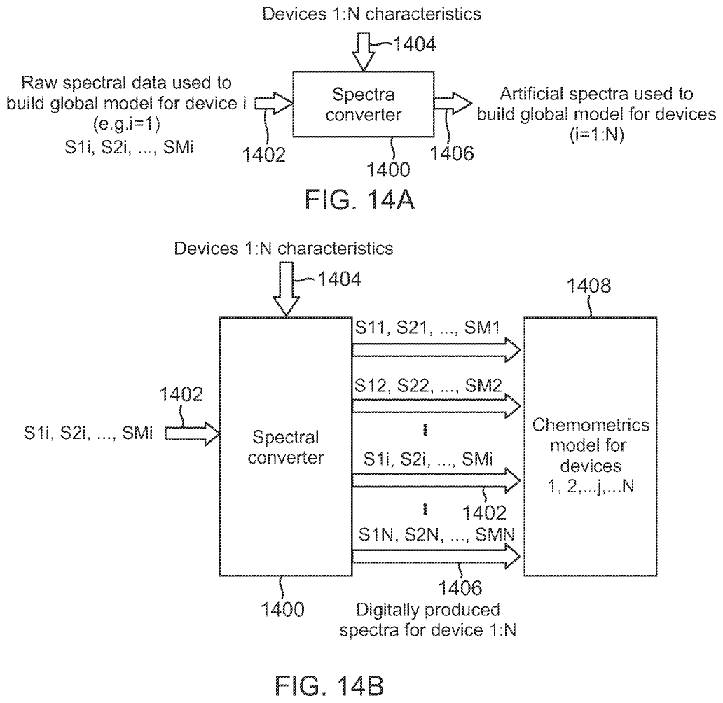

FIG. 13 is a diagram illustrating another example of a spectral modeling system 1300 for two spectral devices 1302 and 1304 according to some aspects. In the example shown in FIG. 13, universal extraction samples 1306 are used. These samples 1306 are not model-specific. As a result, if the chemometrics model is for certain types of feed samples, for example hay, the universal samples 1306 are not part of the hay samples used for building the model. In this case, the universal extraction samples 1306 are measured on the production line of the spectral devices 1302 and 1304. In the example shown in FIG. 13, the universal samples 1306 are measured on a first spectral device (Spectral Device 1) 1302 and a second spectral device (Spectral Device 2) 1304. The measured spectra 1308a and 1308b of the universal samples 1306 are fed to a characteristics extractor 1310 that conducts mathematical calculations to extract the characteristics of the spectral devices 1302 and 1304. These spectral device characteristics 1312 are then fed to a spectral converter 1316 for conversion of the spectra (spectral data) 1314 of a plurality of samples (e.g., M samples), which may correspond to feed samples or other types of samples, measured by the first spectral device 1302 (e.g., S11, S21, . . . , SM1) to the expected spectra (e.g., artificial spectra)1318 for the second spectral device 1304 (e.g., S12, S22, . . . , SM2).

Given that the characteristics of any spectral device (e.g., spectral sensor or spectral scanning device) can be extracted, as discussed above, the characteristics can be used to convert the spectra measured by one spectral device to the expected spectra from another spectral device. FIGS. 14A and 14B are diagrams illustrating an example of a global spectral converter 1400 according to some aspects. In each of FIGS. 14A and 14B, the global spectral converter 1400 is fed by raw spectral data 1402 of a plurality of samples (e.g., M samples) measured by a subset of a plurality of spectral devices (S S2i, . . . , SMi, where i refers to the spectral device). The spectral converter 1400 is also fed by the spectral device characteristics 1404 of N devices (Devices 1:N characteristics). The spectral converter 1400 produces a plurality of converted (artificial) spectra 1406 (S11, S12, S21, . . . , SM1 . . . S1N, S2N, . . . , SMN) for the N spectral devices. Thus, the output of the spectral converter 1400 is N times the input of the spectral converter 1400. The produced artificial spectra 1406, along with the original raw spectral data 1402, can be then used to build a global chemometrics model 1408 that may be used by each of the N devices, as shown in FIG. 14B.

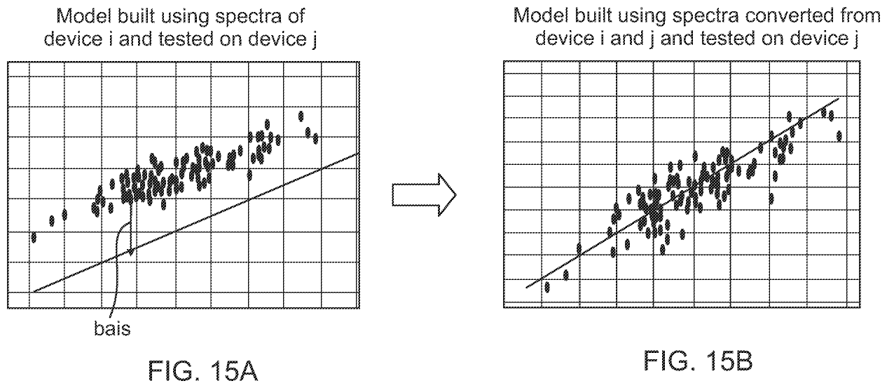

FIGS. 15A and 15B are graphs representing the impact on the bias and slope errors in the prediction according to some aspects. FIG. 15A illustrates the bias and slope errors for a chemometrics model using spectra of a first spectral device (device i) that is tested on a second spectral device (device j), while FIG. 15B illustrates the bias and slope errors for a chemometric model using spectral data converted from device i based on the spectral device characteristics of device j (e.g., as shown in FIGS. 14A and 14B) and then tested on device j. FIG. 15B shows a bias enhancement using the global spectral converter of FIGS. 14A and 14B.

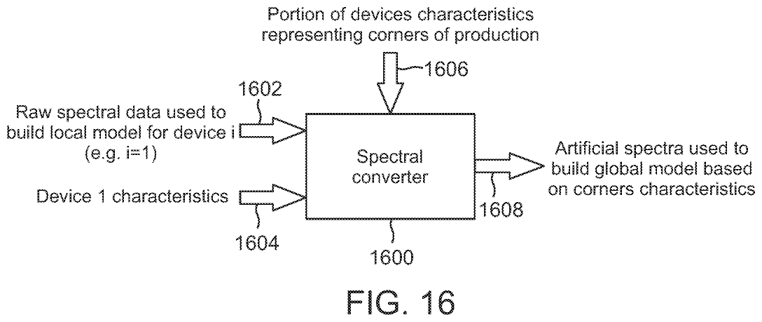

FIG. 16 is a diagram illustrating another example of a global spectral converter 1600 according to some aspects. In the example shown in FIG. 16, the spectral converter 1600 is not fed with the characteristics of the whole N devices. Instead, a portion of the spectral devices are selected and the characteristics 1606 of the selected portion of spectral devices are input to the spectral converter 1600. The portion of spectral devices may be selected such that their characteristics 1606 cover a space of variations including corners of production line characteristics (e.g., of the manufacturing process) of the production line of the plurality of spectral devices. Thus, the portion of the spectral devices may have characteristics 1606 that cover the space of variations including corners of production line characteristics.

In the example shown in FIG. 16, the spectral converter 1600 is further fed by the raw spectral data 1602 of a plurality of samples (e.g., M samples) measured by a single spectral device (device i), along with the spectral device characteristics 1604 of device i. Based on the spectral device characteristics 1604 and 1606 and the spectral data 1602, the spectral converter 1600 can produce the artificial spectra 1608 corresponding to the selected (corner) spectral devices. These artificial spectra can then be used to build a global chemometrics model for all the spectral devices of production line based on the process corners. In some examples, the corner spectral devices can be selected using a stable materials mixture. This mixture may represent the actual materials that are intended to be measured. By measuring this mixture by all spectral devices on the production line and building a clustering model of spectral devices, the corner spectral devices that represent the population of spectral devices can be selected.

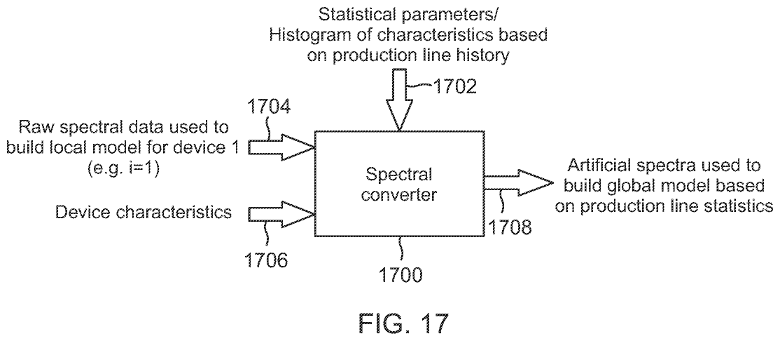

FIG. 17 is a diagram illustrating another example of a global spectral converter 1700 according to some aspects. In the example shown in FIG. 17, the spectral device characteristics fed to the spectral converter 1700 are not actual measured ones for all spectral devices or even selected spectral devices. Instead, the spectral device characteristics 1702 are generated characteristics based on an understanding of the production line variations and histograms. For example, with knowledge of statistical information related to the production line history, various statistical parameters may be derived. For example, the mean value and the standard deviations of the characteristics can be ascertained. Other statistical parameters, such as the skewness and/or kurtosis can be concluded. In addition, the probability distribution (e.g., a normal, double exponential, Cauchy, Weibull, or other distribution) of each of the statistical parameters of the characteristics can be also determined. The probability distribution (e.g., histogram), together with the statistical parameters, such as the mean, standard deviation, skewness and/or kurtosis can then be used to generate the representative characteristics 1702 that are fed to the spectral converter 1700.

The spectral converter 1700 is further fed by the raw spectral data 1704 of a plurality of samples (e.g., M samples) measured by a single spectral device (device i), along with the spectral device characteristics 1706 of device i. Based on the spectral device characteristics 1702 and 1706 and the spectral data 1704, the spectral converter 1700 can produce the artificial spectra 1708 that may be used to build a global chemometrics model based on the production line statistics. In this example, the artificial spectra 1708 generated by the spectral converter 1700 does not correspond in a one-to-one manner to any of the spectral devices produced on the production line. However, the artificial spectra 1708 does correspond to virtual spectral devices that span across the characteristics of the production line.

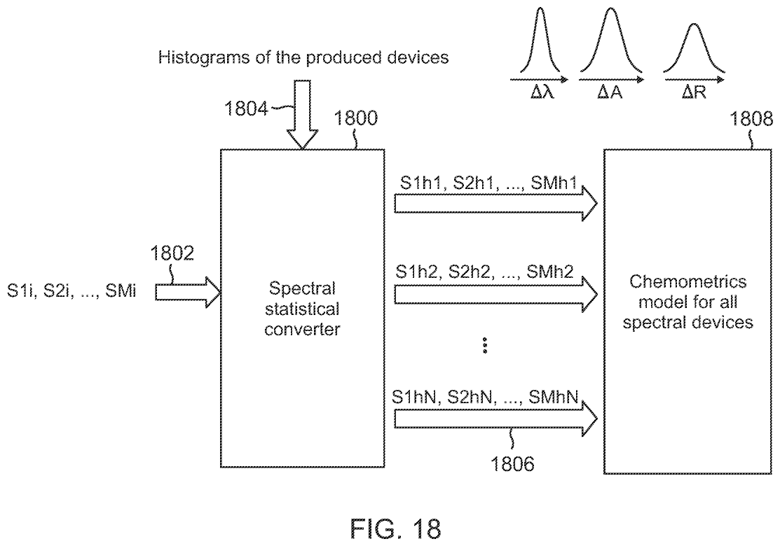

FIG. 18 is a diagram illustrating another example of a global spectral converter 1800 according to some aspects. The global spectral converter 1800 shown in FIG. 18 is a global statistical spectral converter fed by the spectral data 1802 of the samples (e.g., M samples) measured by a subset of a plurality of spectral devices (S1i, S2i, . . . , SMi, where i refers to the spectral device). The spectral statistical converter 1800 is further fed by the statistical spectral device characteristics (e.g., histogram or probability distribution) 1804 of the production line spectral devices. The spectral statistical converter 1800 produces the artificial spectra 1806 (S1h1, S2h1, SMh1 . . . S1hN, S2hN, . . . , SMhN) based on the spectral data 1802 and the statistical spectral device characteristics 1804. As in the example shown in FIG. 17, the artificial spectra 1806 does not correspond one-to-one with any of the production line devices, but instead represent the spectra for hypothetical spectral devices with combinations of characteristics. The produced artificial spectra 1806 can be then used to build a global chemometrics model 1808 for all spectral devices. This chemometrics model 1808 is global in the statistical sense, but not necessarily corresponding to real spectral devices from the production line.

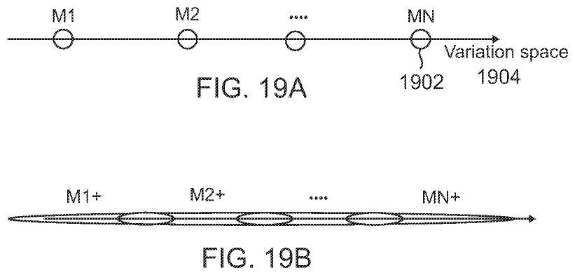

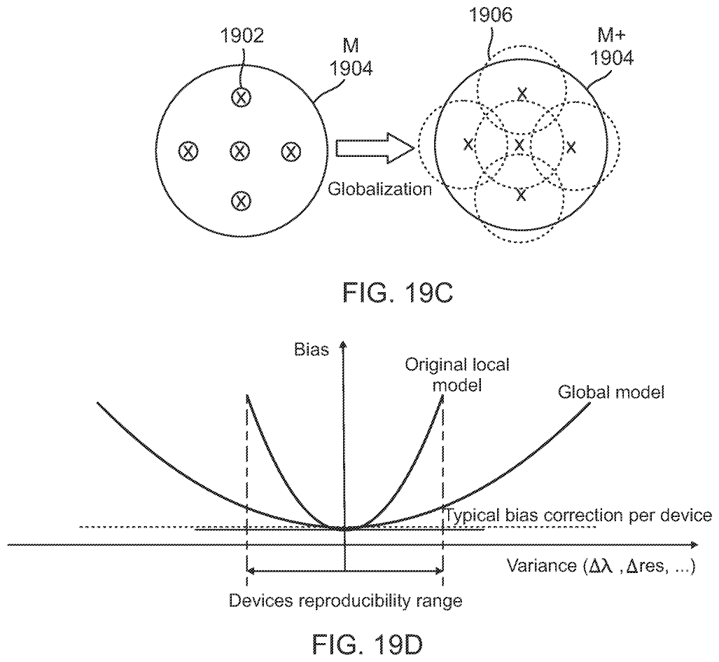

In some examples, the spectral converter can apply pre-processing on the spectral data provided by the subset of spectral devices, as shown in FIGS. 19A-19D. The subset of spectral devices (e.g., M spectral devices) can be projected on the production line variations space 1904, as shown in FIG. 19A. The chemometrics model coverage of each spectral device represents a discrete point 1902 in the space 1904. To cover the whole space 1904, the spectral converter can add modulation or perturbation around each discrete point 1902 in the corresponding dimension. Thus, each spectral device M becomes a region in the space M+. A two-dimensional representation of the variation spaces M and M+ are shown in FIG. 19C. The concatenation of the different modulated spectral devices leads to coverage of the whole space within the region of interest corresponding to the production line devices, which can lead to an improvement of prediction bias sensitivity to spectral device reproducibility as compared to the original local chemometrics model for a single spectral device, as shown in FIG. 19D.

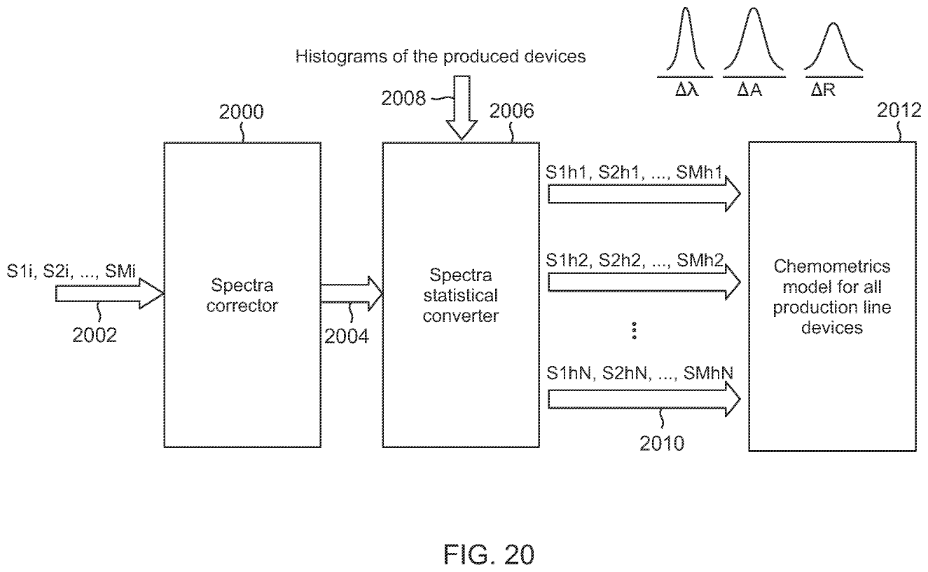

FIG. 20 is a diagram illustrating another example of a global spectral converter according to some aspects. In the example shown in FIG. 20, a spectral corrector 2000 is used to clean and remove uncontrolled variations in one or more of the specifications (spectral device characteristics) of the subset of spectral devices on which measured spectral data for plurality of samples (e.g., M samples, corresponding to S1i, S2i, . . . , SMi, where i refers to the spectral device) is obtained. For example, there may be outliers in the specifications or the specifications may not be quantified and extracted on the production line. In other examples, a specification may be measured on the production line for the subset of spectral devices, but the subset of spectral devices may not be well distributed in the space with respect to this specification. Whatever the value of the specification, which can be unknown, the spectral corrector 2000 can apply a spectral variance function (mathematical operation) to the spectral data 2002 to unify all of the subset of spectral devices in terms of this specification.

The resulting processed spectral data 2004 may be fed into a spectral statistical converter 2006 that is further fed by the statistical spectral device characteristics (e.g., histogram or probability distribution) 2008 of the production line spectral devices. The spectral statistical converter 2006 produces the artificial spectra 2010 (S1h1, S2h1, SMh1 . . . S1hN, S2hN, . . . , SMhN) based on the processed spectral data 2004 and the statistical spectral device characteristics 2006. The produced artificial spectra 2010 can be then used to build a global chemometrics model 2012 for all spectral devices.

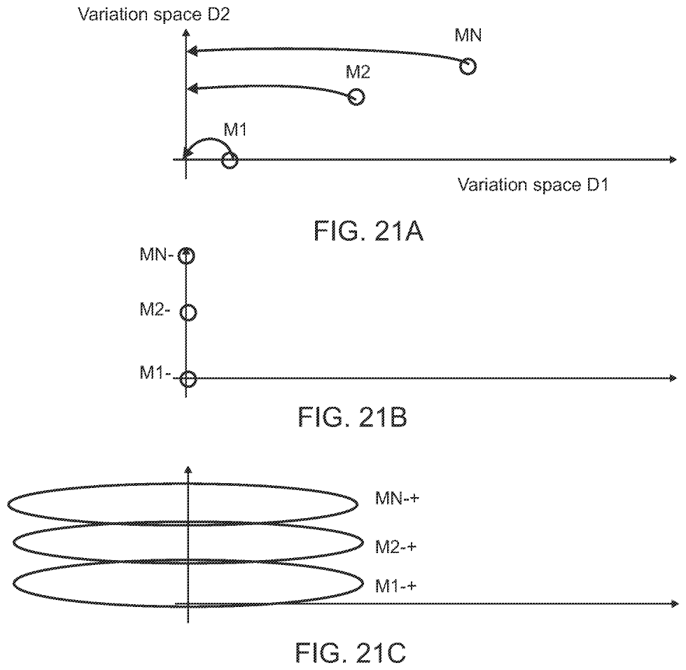

FIGS. 21A-21C are diagrams illustrating an example of spectral correction according to some aspects. As shown in FIG. 21A, specification variations between the subset of spectral devices (M) can be in one of the dimensions (D1 or D2) of the variation space. The spectral corrector can remove the variation (by applying the spectral variance function) on the spectral data produced by the M spectral devices (e.g., M1, M2, and M3) to unify the spectral devices (M−) in the D1 dimension, as shown in FIG. 21B. As further shown in FIG. 21C, the spectral statistical converter can add controlled variations on the spectral data (e.g., based on the statistical spectral device characteristics) to cover the whole space of variations expected from the production line of the spectral devices, denoted as M−+ to indicate the two steps (spectral correction and spectral conversion) applied. In this example, the modulation on the D2 axis is small, since the M− devices are distributed on this axis. The modulation is double sided on the D1 axis in the space.

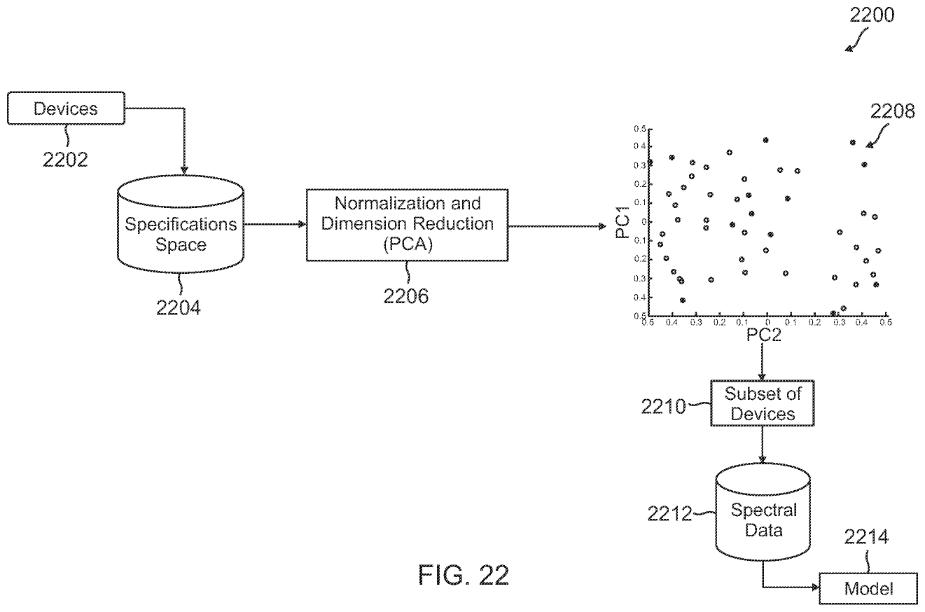

FIG. 22 is a diagram illustrating an example of an algorithm 2200 for selection of the subset of spectral devices to collect the spectral data according to some aspects. Randomly choosing the subset of spectral devices may affect the chemometrics model's accuracy, and its accuracy may vary from one spectral device to another. Therefore, by appropriately selecting the subset of spectral devices to include as much as possible the variations of the spectral device characteristics, the chemometrics model can be trained on such variations in advance, which will standardize its accuracy across all spectral devices.

In the example shown in FIG. 22, the algorithm may form a specifications space 2204 from all the available spectral devices 2202. Due to the large size of the data and the diverse nature of the data, dimension reduction and data normalization 2206 can then be applied to the specification space data before visualization. Many techniques perform dimension reduction, such as principal component analysis (PCA), which reduces the data dimensionally while reserving its variations. The number of principal components (PC) can be determined according to the reserved variations inside them, which in some examples, can be more than 95% of the total variations in the original data. The PCs can then be plotted, as illustrated in FIG. 22. The plot shown in FIG. 22 includes two PCs for simplicity. However, it should be understood that the number of PCs usually exceeds two. The algorithm can then select the subset of spectral devices 2210 such that the selected spectral devices are distributed to have a portion around the mean of the PCs and the other part on the extremes. Gaussian maximum likelihood estimation MLE can further be applied on the data to make a model for the spectra position from the center of variations on the multi dimension space. Spectral devices can be ranked based on distance from center, with spectral devices at the center being the most reproducible, and spectral devices at the edges being the least reproducible. Mahalanobis distance can be used to calculate this distance. The chosen (selected) subset of spectral devices may then be used to collect the spectral data 2212 that is used to build the chemometrics model 2214.

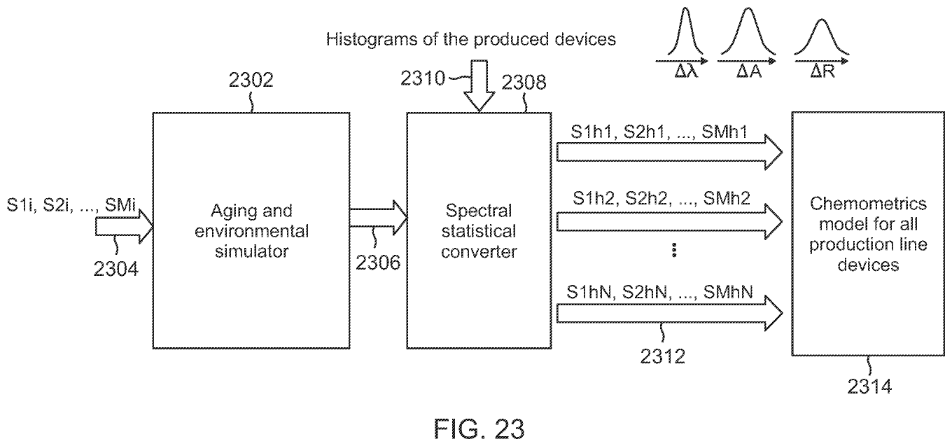

FIG. 23 is a diagram illustrating another example of spectral correction according to some aspects. In the example shown in FIG. 23, an aging simulator and environmental effects simulator processor 2302 is fed the raw spectral data 2304 of a plurality of samples (e.g., M samples) measured by a subset of spectral devices (S1i, S2i, . . . , SMi) and applies a spectral modulation and perturbation function that removes uncontrolled variances in the subset of spectral devices to produce processed spectral data 2306. For example, the spectral modulation and perturbation function may modulate and adds perturbation to the spectral data and convert the spectral data to new processed spectral data that spans different levels of environmental effects, such as temperature variations, humidity levels, vibrations and alike. Aging effects, such as accumulation of humidity, degradation in the light source intensity and alike can also be added.

The processed spectral data 2306 and spectral device characteristics 2310 (e.g., histograms of the produced spectral devices) may then be fed into a spectral statistical converter 2308 to produce the artificial spectra 2312 (S1h1, S2h1, SMh1 . . . S1hN, S2hN, . . . , SMhN) for hypothetical spectral devices with combinations of characteristics 2310 and aging with time. The artificial spectra 2312 may then be used to build the chemometrics model 2314 for all production line spectral devices. By adding modulation and perturbation to the spectral data to account for environmental and aging effects, the resulting chemometrics model 2314 may be self-maintained, and may not need updates from time to time to account for the aging of the devices or seasonal changes in the environment.

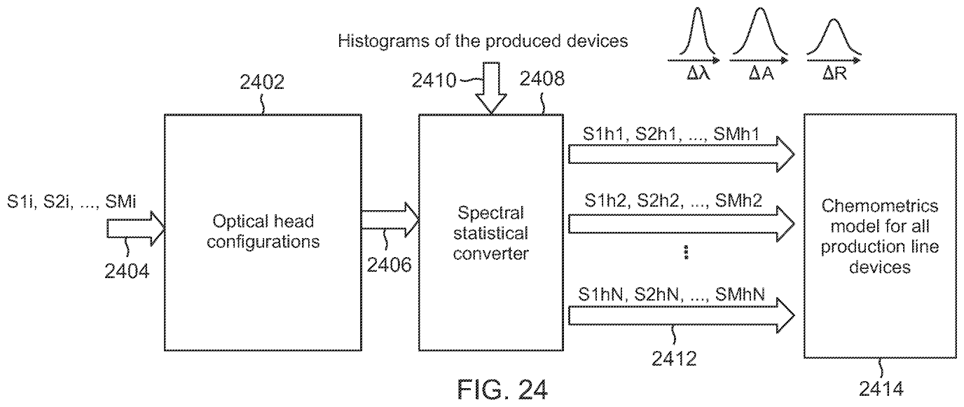

FIG. 24 is a diagram illustrating another example of spectral correction according to some aspects. In the example shown in FIG. 24, an optical head configurations processor 2402 is included to account for variants in the optical head configurations of the spectral devices. The optical head configurations processor 2402 may be used, for example, to simulate the effect of different optical head configurations by applying an optical head variance function to the raw spectral data 2404 of a plurality of samples (e.g., M samples) measured by a subset of spectral devices (S1i, S2i, . . . , SMi) to produce processed spectral data 2406.

The variation can be in the spot size of the illumination/collection, distance between the sample and the optical window, the use of multiple optical windows, the use of sample rotator averaging spatial in homogeneity in the sample and alike. Different mathematical operations associated with the optical head variance function can be used to account for such variants, such as the adjustment of the baseline by different orders, the application of differential spectroscopy techniques, averaging multiple spectra from different fills of the sample, and/or other mathematical operation.

The processed spectral data 2406 and spectral device characteristics 2410 (e.g., histograms of the produced spectral devices) may then be fed into a spectral statistical converter 2408 to produce the artificial spectra 2412 (S1h1, S2h1, SMh1 . . . S1hN, S2hN, . . . , SMhN) for hypothetical spectral devices with combinations of characteristics 2310 and aging with time. The artificial spectra 2412 may then be used to build the chemometrics model 2414 for all production line spectral devices. Therefore, the use of one optical head configuration and the use of the optical head configurations processor 2404 can lead to generalizing the chemometrics model 2414 for other optical head configurations.

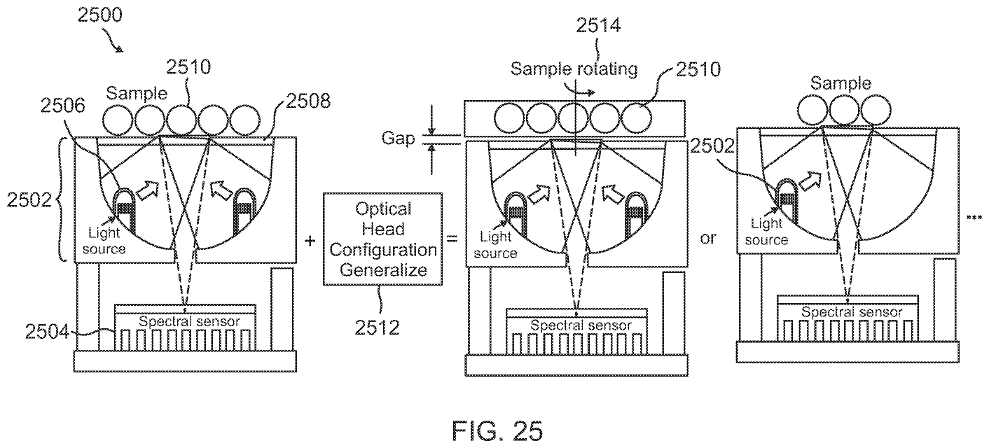

FIG. 25 is a diagram illustrating examples of optical head configuration generalization according to some examples. FIG. 25 illustrates an example of a spectral device 2500 including an optical head 2502 and a spectral sensor 2504 (e.g., light modulator and detector). The optical head 2502 may include a plurality of light sources 2506 configured to generate incident light that is directed towards a transparent window 2508 of the optical head 2502 to interact with a sample 2510 on the top surface of the transparent window 2508. An optical head configuration generalizer 2512 (e.g., which may correspond, for example, to the optical head configurations processor 2402 shown in FIG. 24) can apply an optical head variance function to spectral data produced by the spectral device to account for variations in the optical head 2502. For example, the optical head configuration generalizer 2512 may account for scanning/rotating mechanisms 2514 for rotating the sample 2510, different number of light sources 2502, different optical spot sizes, different sampling distances, or other variations in the optical head configuration.

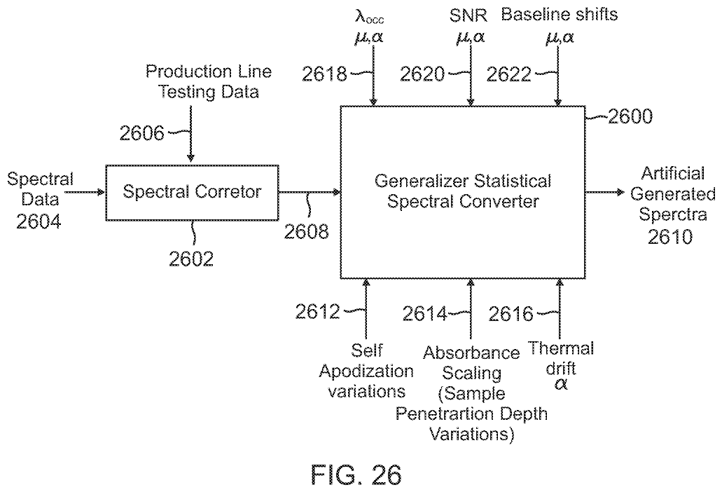

FIG. 26 is a diagram illustrating another example of spectral correction according to some aspects. To be able to cover the variations of spectral performance of high-volume spectral devices, a generalizer statistical spectral converter 2600 can be used to generate artificial spectra of many spectral devices from one or a subset of the spectral devices. In some examples, the subset of spectral devices can be selected to be of high performance, with minimal wavelength error, minimal resolution error, minimal photometric error, high SNR and minimal baseline shifts and minimal thermal drift effects. In other examples, as shown in FIG. 26, the spectral data 2604 produced by the subset of spectral devices may first be corrected by a spectral corrector 2602 based on production line testing data 2606 to produce processed spectral data 2608.

The generalizer statistical spectral converter 2600 is configured to convert the spectral data 2608 to a plurality of artificial generated spectra 2610. The spectral conversion process performed by the generalizer statistical spectral converter 2600 may include multiple spectral effects based on the spectral device characteristics being applied onto the spectral data 2608, as shown in FIG. 26. For example, the generalizer statistical spectral converter 2600 may apply one or more spectral effects to the processed spectral data 2608, including, for example, self-apodization variations, absorbance scaling 2614, thermal drift (wavelength α dependent) 2616, wavenumber/wavelength errors λ_{occ} across the range (μ,α) 2618, SNR across the range (μ,α) 2620, and/or baseline shifts across the range (μ,α) 2622.

Selection of the spectral effects to be applied, the amount of variations, and the order of the spectral effects, can be optimized by the generalizer statistical spectral converter 2600 for each application to minimize the bias between different spectral devices and their respective prediction errors. The added effects can be due to variations related to the photodetector response, light source, optical coupling elements between the light source, interferometer and photodetector, background samples, distance between the sample and the optical windows, actuation and sensing electronics. The effects can be experimentally extracted, empirically fitted or modeled behaviorally based on physical effects in the form of compact models.

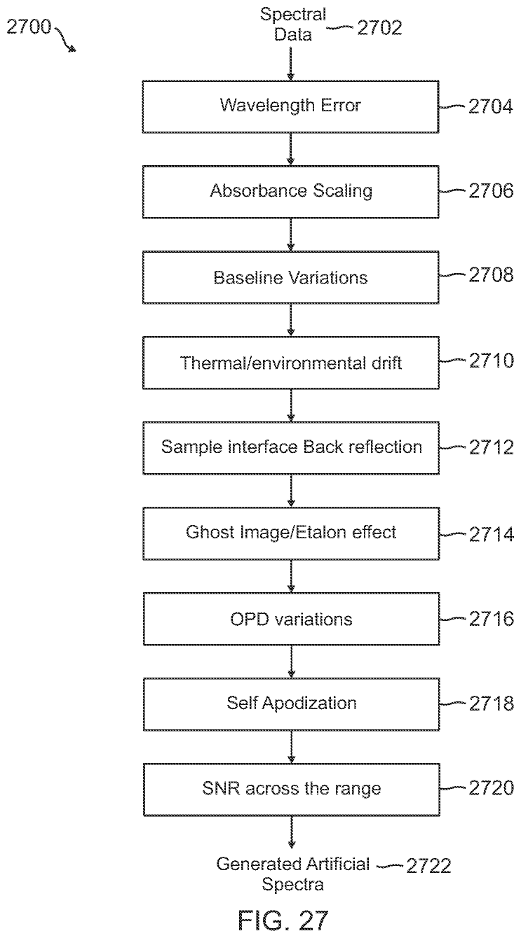

FIG. 27 is a diagram illustrating an example of a process flow for generating artificial spectra according to some aspects. The process flow 2700 may be performed, for example, by the generalizer statistical spectral converter 2600 shown in FIG. 26. The input to the process flow 2700 includes spectral data obtained by a subset of a plurality of spectral devices. The process flow 2700 applies a plurality of spectral effects to the spectral data to produce (generate) artificial spectra 2722. For example, at block 2704, wavelength errors may be added to the spectral data 2702. At block 2706, an absorbance spectrum of the spectral data may be scaled using a wavelength dependent scaling factor. At block 2708, baseline variations may be added to absorbance of the spectral data. At block 2710, the spectral data may be multiplied by a thermal/environmental drift factor across wavelengths. At block 2712, sample interface back reflection spectra may be added to the spectral data. At block 2714, the spectral data may be multiplied with a ghost image/Etalon effect. At block 2716, optical path difference (OPD) errors may be applied to the spectral data. At 2718, a set of apodization functions may be applied to the spectral data to account for self-apodization variations. At 2720, noise across a spectral range corresponding to a SNR distribution may be applied to the spectral data.

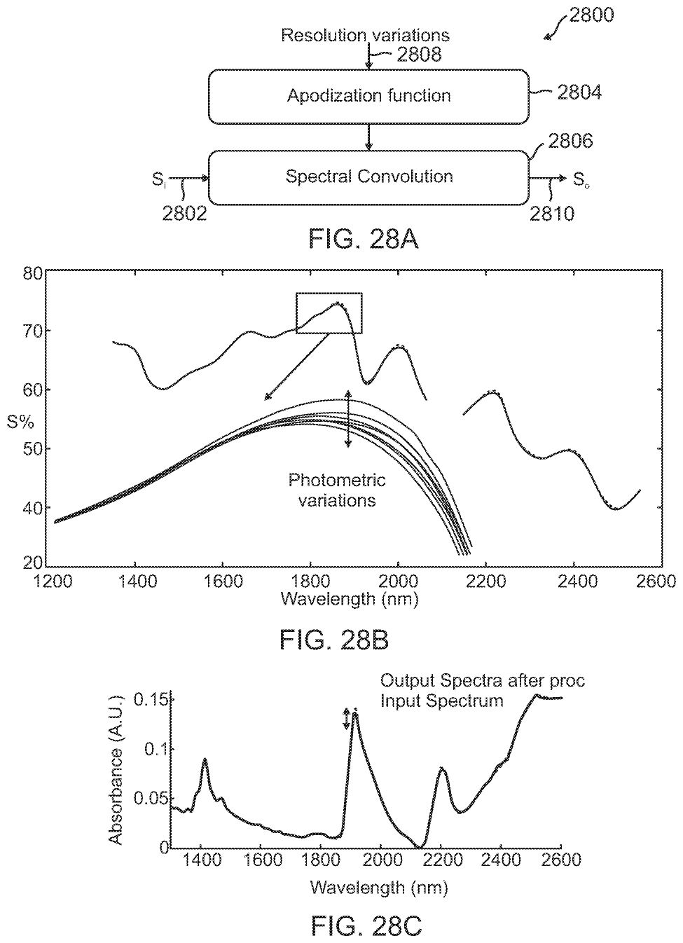

FIGS. 28A-28C illustrate an example of generalization of spectral data based on self-apodization and resolution variations according to some aspects. FIGS. 28A-28C may correspond to block 2718 shown in FIG. 27. FIG. 28A is a diagram illustrating a spectral converter 2800 for application of a set of apodization functions 2804 (e.g., spectral device characteristics) to spectral data (Sᵢ) 2802 according to some aspects. Apodization functions 2804 correspond to self-apodization effects that can vary from spectral device to spectral device. A spectral convolution processor 2806 extracts the interferograms from the input spectral data 2802 and multiplies the extracted interferograms by stored self-apodization functions 2804 that are based on spectral resolution variations 2808 (e.g., resolution changes) covering a range of resolution distributed across the plurality of artificial spectra. Thus, the spectral convolution processor 2806 may convolute the input spectral data 2802 with a set of apodization functions 2804 to produce the output artificial spectra (Sₒ) 2810. The self-apodization effect is generally wavelength dependent, so the spectral data 2802 can be convoluted with wavelength dependent apodization functions 2804 that represent the variation of self-apodization across the spectral range.

For example, inputs to the spectral converter 2800 may include the transmission or reflection spectral data (Sᵢ) to be processed, the background PSD (PSD_{background}), and resolution change 2808. The spectral converter 2800 may extract the interferogram from the spectral data as follows:

PSDp=Sᵢ×PSD_{Background} (7)

Interferogram=inverse FT(PSDp)=Iₒ (8)

The spectral converter 2800 may then choose an apodization functions set 2804 based on the resolution change values 2808 and multiply the extracted interferogram by the set of apodization functions 2804 to produce the artificial spectra (Sₒ) 2810 as follows:

$${I}_{n e w} = {I}_{o} \times Apodization function \qquad ( 9 )$$

$${S}_{o} = \frac{F T ( {I}_{n e w} )}{P S {D}_{B a c k g r o u n d}} \qquad ( 10 )$$

The spectral converter 2800 changes the spectral resolution and accordingly the photometric accuracy of the spectral data 2802 by apodization of the corresponding interferograms of the input spectral data 2802, as shown in FIGS. 28B and 28C.

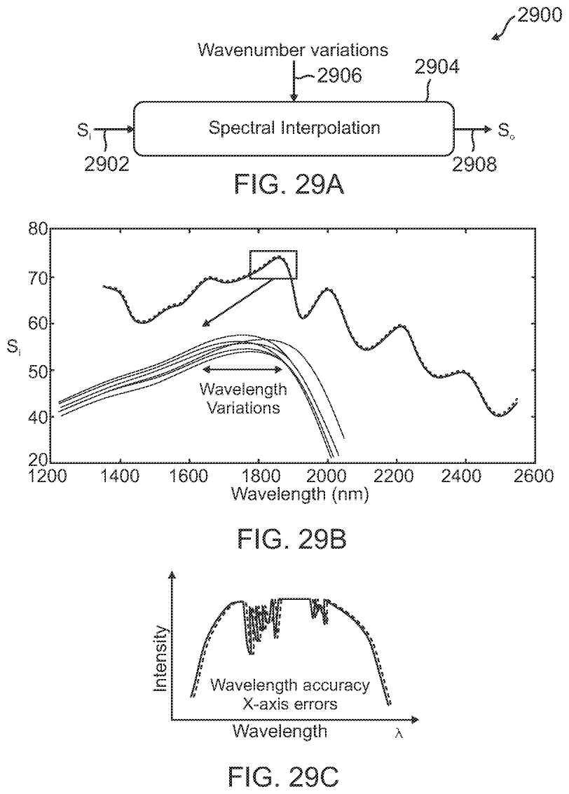

FIGS. 29A-29C illustrate an example of generalization of spectral data based on wavenumber/wavelength errors according to some aspects. FIGS. 29A-29C may correspond to block 2704 shown in FIG. 27. FIG. 29A is a diagram illustrating a spectral converter 2900 for application of wavenumber/wavelength errors (e.g., spectral device characteristics) to spectral data (Sᵢ) 2902 according to some aspects. The spectral converter 2900 may be configured to add wavelength errors to the input spectral data 2902 in different forms. Inputs to the spectral converter 2900 may include the input spectral data 2902 to be processed and wavenumber variations 2906, which may include, for example, gain error standard deviation and offset error standard deviation.

The wavenumber vector can be shifted from the original input wavenumber vector as follows:

ν_{new}=(1+Gain error)νᵢₙ+Offset error (11)

Wavelength error can vary from wavelength position to wavelength position along the spectrum, as shown in FIG. 29B, and therefore, is a non-linear trend that may happen due to errors in calibration of retardation or the off-axis movements of the moving mirror or other non-linear interference/diffraction effects. To account for higher order wavelength errors, a higher order correction function can be used as follows:

ν_{new}=aₙνᵢₙⁿ+aₙ₋₁νᵢₙⁿ⁻¹a₁νᵢₙ+Offset error (12)

The output artificial spectra (Sₒ) 2908 can be generated through interpolation of the shifted spectra via spectral interpolation block 2904 onto the input wavenumber vector, as shown in FIG. 29C.

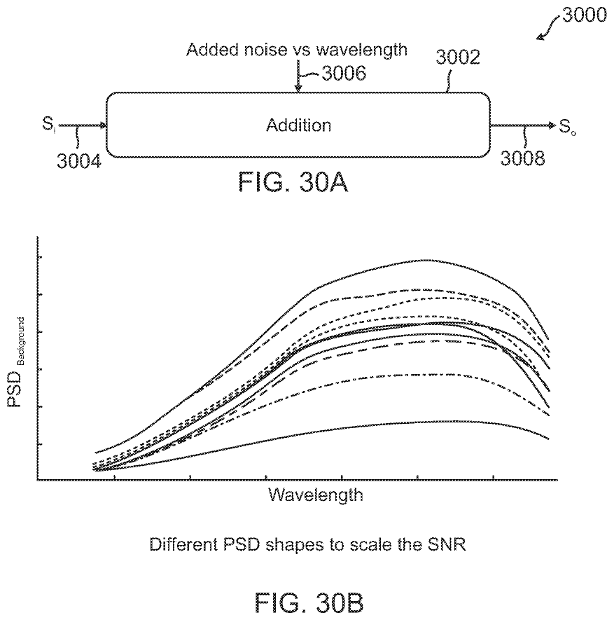

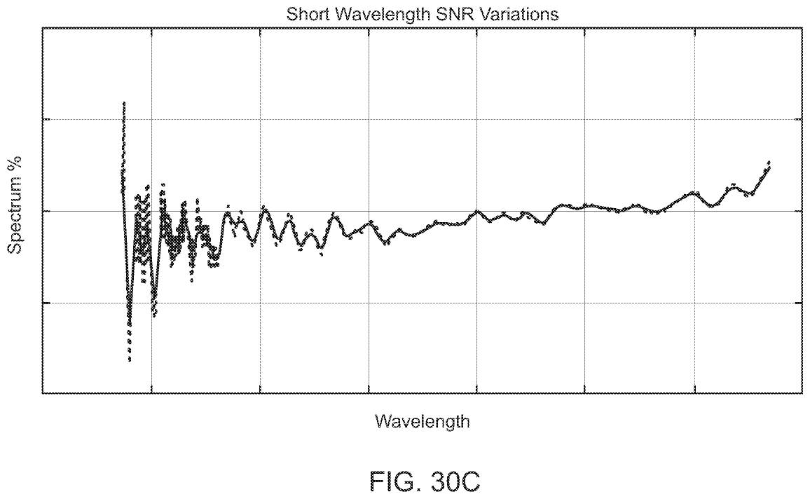

FIGS. 30A-30C illustrate an example of generalization of spectral data based on SNR across the wavelength range according to some aspects. FIGS. 30A-30C may correspond to block 2720 shown in FIG. 27. FIG. 30A is a diagram illustrating a spectral converter 3000 for decreasing the SNR of the spectral data according to some aspects. For example, the spectral converter 3000 can directly decrease the SNR with specific value(s) based on the spectral device characteristics. On the production line, spectral devices may have different SNRs with mean SNR and standard deviation. Inputs to the spectral converter 3000 may include the input spectral data Sᵢ 3004 to be processed, the SNR of the subset of spectral devices and new (target) SNR(s) (mean and standard deviation), which may correspond to the spectral device characteristics. The noise standard deviation 3006 can then be calculated as:

$$Noise σ = max ( {PSD}_{B a c k ground} ) * \sqrt{\frac{1}{S N {R}_{n e w}^{2}} - \frac{1}{S N {R}_{orginal}^{2}}} \qquad ( 13 )$$

The noise standard deviation can then be added to the input spectral data Sᵢ 3004 by addition block 3002 to produce output artificial spectra Sₒ 3008 as follows:

$${S}_{o , noisy} = {S}_{i} + 10 \; 0 * \frac{Noise}{{PSD}_{Background}} \qquad ( 14 )$$

Different shapes of PSD_{Background} covering device to device variations, as shown in FIG. 30B, can be used to scale the SNR across wavelength. As shown in FIG. 30C, added noise with different PSDs shaping scaling produces different SNR at short wavelengths using different apodization functions.

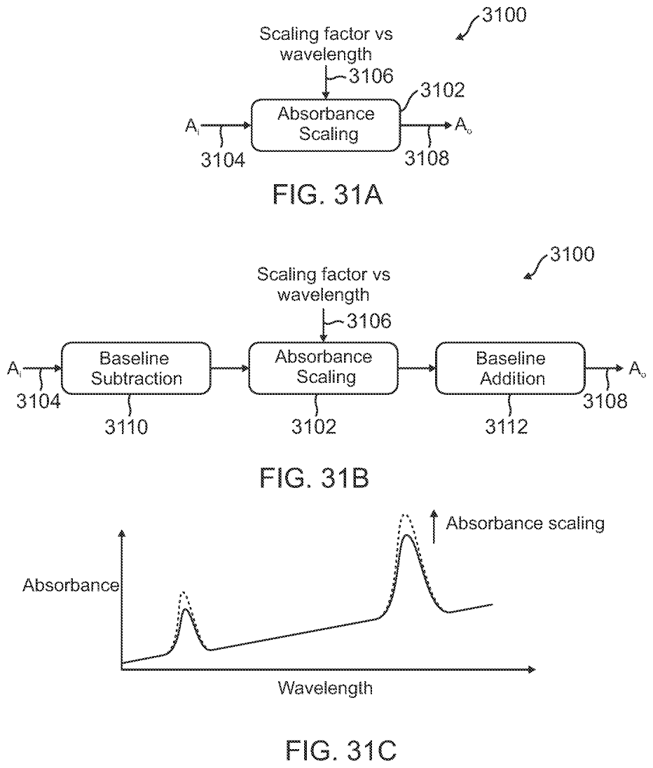

FIGS. 31A-31C illustrate an example of generalization of spectral data based on absorbance scaling according to some aspects. FIGS. 31A-31C may correspond to block 2706 shown in FIG. 27. FIG. 31A is a diagram illustrating a spectral converter 3100 for emulating the variations resulted from penetration depth variations associated with sample to spectral device spacing variations, sample heterogeneity nature, and sampling accessory/method variations, each of which may correspond to spectral device characteristics. In the example shown in FIG. 31A, the spectral converter 3100 may include an absorbance scaling block 3102 configured to scale the whole absorbance spectrum 3104, as shown in FIG. 31C, by a wavelength dependent scaling factor SF(λ) 3106, such that the output absorbance spectrum Aₒ 3108 is given by:

Aₒ=SF(λ)Aᵢ (15)

In other examples, as shown in FIG. 31B, the spectral converter 3100 may scale absorption line intensities by first subtracting the baseline A_{i,BL} through a baseline extraction method performed by baseline subtraction block 3110, adjusted and optimized to the spectra under analysis, and then adding the baseline A_{i,BL} back by a baseline addition block 3112 after scaling. Baseline extraction methods may include, for example, moving average detection techniques, partial least squares PLS-based techniques, wavelet transform techniques, etc. In this example, the output absorbance spectrum 3108 is generated from the input absorbance spectrum 3104 as:

Aₒ=SF(λ)(Aᵢ−A_{i,BL})+A_{i,BL} (16)

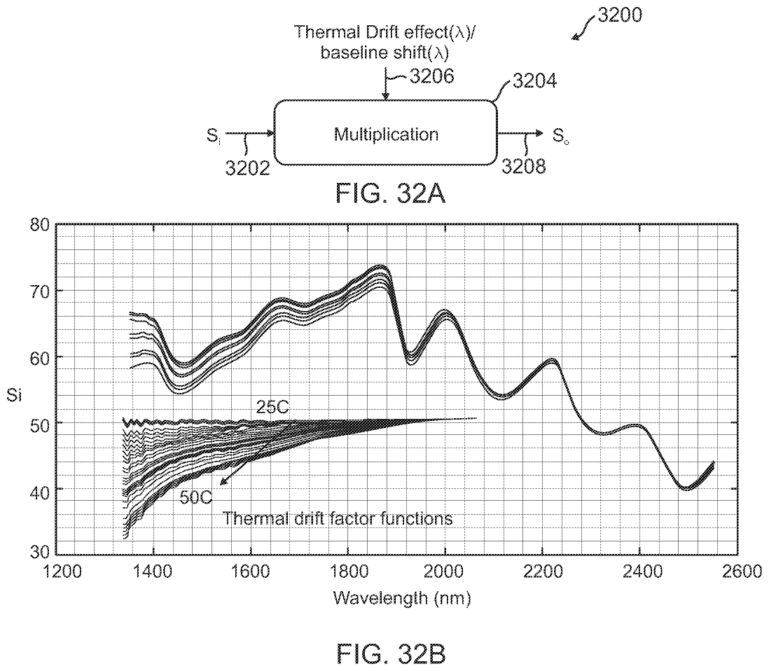

FIGS. 32A and 32B illustrate an example of generalization of spectral data based on thermal drift according to some aspects. FIGS. 32A and 32B may correspond to block 2710 shown in FIG. 27. FIG. 32A is a diagram illustrating a spectral converter 3200 for randomly adding thermal drift effects 3206 to the input spectral data 3202 according to some aspects. The spectral data 3202 Sᵢ can be multiplied by the thermal drift factor across wavelength 3206 via multiplication block 3204. This can include wavelength-dependent baseline shifts as well. Inputs to the spectral converter 3200 include temperature variations with respect to the background measurement temperature (e.g., spectral device characteristics) and the spectral data Sᵢ to be processed.

The resulting generated (artificial) spectra 3208 are related to the input spectral data 3202 as either:

Sₒ=Sᵢ*Rate_{Thermal drift}*(T−T_{BG}) (17)

where Rate_{Thermal drift} is the rate of spectral variations per temperature degree or:

Sₒ=Sᵢ*Response(T)/Response(T_{BG}) (18)

where Response(T) is the system spectral response across temperature, that includes the thermal behavior of different system components.

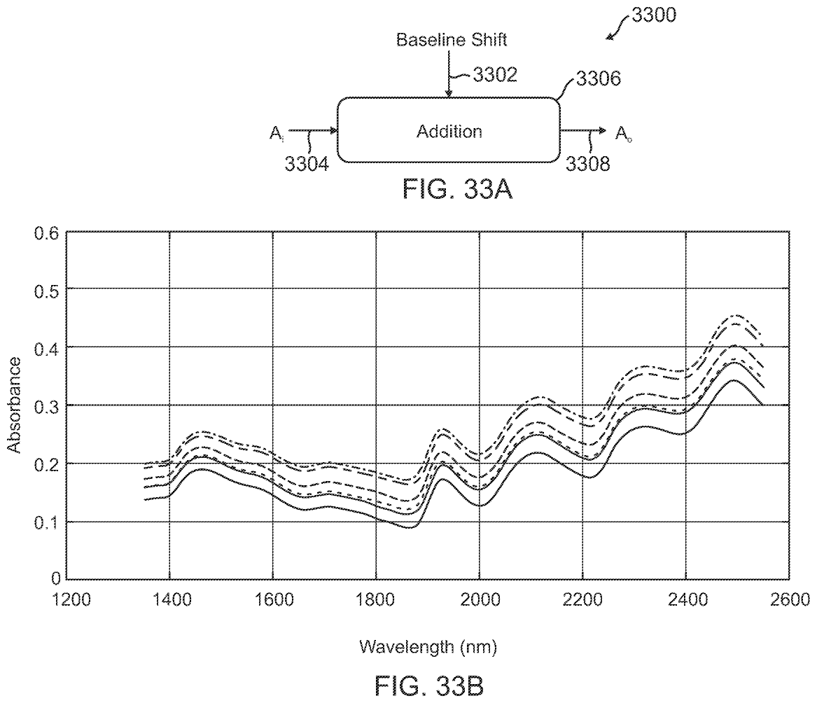

FIGS. 33A and 33B illustrate an example of generalization of spectral data based on baseline shift according to some aspects. FIGS. 33A and 33B may correspond to block 2708 shown in FIG. 27. FIG. 33A is a diagram illustrating a spectral converter 3300 including an addition block 3306 for adding baseline variations 3302 to input absorbance Aᵢ 3304 in different shapes, as shown in FIG. 33B to produce output absorbance Aₒ 3308 according to some aspects. For example, the spectral converter 3300 may add random baseline variations with mean and a standard deviation that scale with wavelength, multiplicative baseline errors that have a higher effect on absorbance peaks, and/or offset baseline variations with mean and a standard deviation. Inputs to the spectral converter 3300 include the spectrums Sᵢ to be processed and various spectral device characteristics, such as baseline absorbance shift E, scaled absorbance error E_{λ} with wavelength, and multiplicative baseline errors that may happens due to samples inhomogeneity E_{scattring}. The output (artificial) spectrum Sₒ can be generated from the input spectrum Sᵢ as follows:

Aᵢ=−log₁₀Sᵢ (19)

Aₒ=Aᵢ*E_{scattring}+E_{λ}*λ+E (20)

Sₒ=10^{-A}^{ₒ} (21)

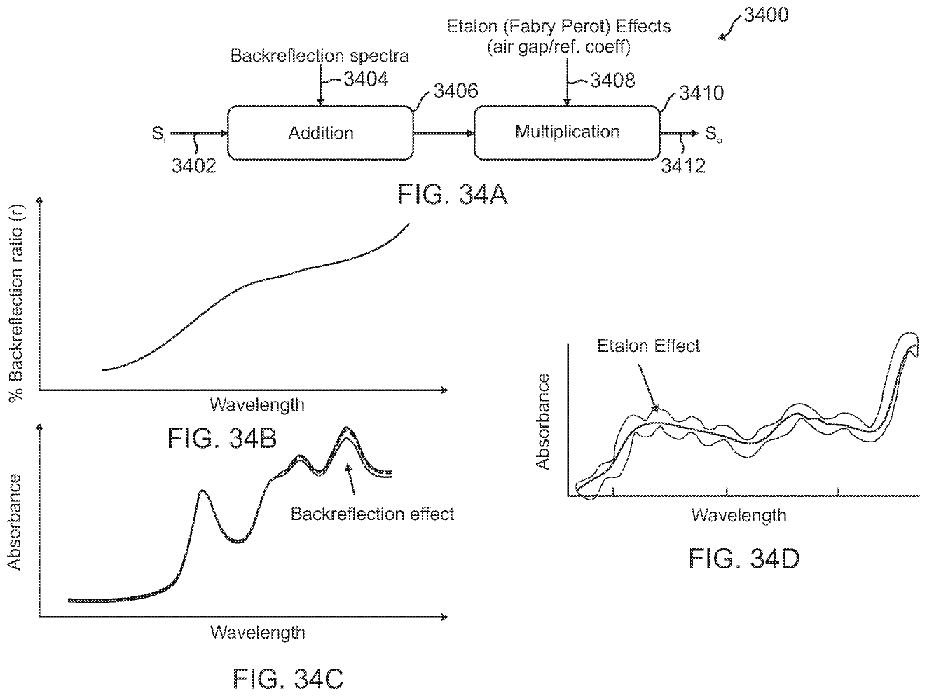

FIGS. 34A-34C illustrate an example of generalization of spectral data based on sample interface effects (e.g., window back reflection and Etalon fringe) according to some aspects. FIGS. 34A-34C may correspond to blocks 2712 and 2714 shown in FIG. 27. FIG. 34A is a diagram illustrating a spectral converter 3400 for emulating sample interface effects according to some aspects. Sample interface effects may include, for example, spectral device window back reflection (e.g., the transmission coefficient never reaches unity and there is always finite reflection from the window that adds to the measured spectrum), as shown in FIGS. 34B and 34C, and/or Etalon fringes associated with samples inside of a container or bag, as shown in FIG. 34D.

As shown in FIG. 34A, the spectral converter 3400 can include an addition block 3406 configured to add back reflection spectra 3404 to the input spectral data Sᵢ 3402 to generate the artificial spectra Sₒ 3412. For example, the back reflection effect can be applied to the input reflectance spectrum to generate the output reflectance spectrum as:

Sₒ=(1+r_{BR}(λ))Sᵢ−r_{BR}(λ) (22)

where r_{BR} is the backreflection ratio of the background spectrum.

The spectral converter 3400 may further include a multiplication block 3410 configured to multiply an Etalon (Fabry Perot) effect 3408 (e.g., air gap/reference coefficients) to the spectral data Sᵢ 3402 to generate the artificial spectra Sₒ 3412. For example, the Etalon effect can be further applied as:

Sₒ=T_{Etalon}(λ)((1+r_{BR}(λ))Sᵢ−r_{BR}(λ)) (23)

Typically, materials that are used for background measurements in bench top spectral devices are calibrated and chosen to be of high purity and stability and consequently of high cost. Moreover, they are kept away from any contamination. However, it is not practical to make such high-purity background materials for each handheld spectral device that are produced on a large scale. Hence, cheap and less pure background materials may be used for handheld spectral devices. Moreover, as handheld spectral devices are meant to be used in the field, it may not be possible to keep them away from contaminations. Therefore, background material for handheld spectrometers may have some variations that may affect model performance.

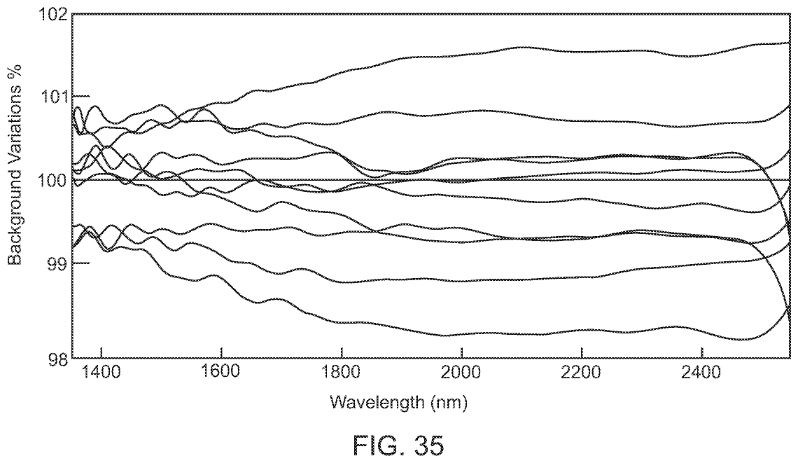

FIG. 35 is a graph illustrating an example of high-reflectance background reference materials variations that may also to be fed to the spectral converter to decrease its susceptibility to such variations in the spectral device characteristics (e.g., generated based on measurements of background reference materials) according to some aspects. For example, an artificial spectrum S_{artf} can be generated from a measured spectrum Sₘₑₐₛ using the background material reflectance variations Rν (λ) as follows:

S_{artf}=Rν(Δ)·Sₘₑₐₛ (24)

Multiple artificial spectra can be generated from one measured spectrum using a set of Rν (λ) spectral variations that covers the variations of the background reference material.

In FTIR spectrometers, the maximum movement distance of the movable mirror or what is known as full travel range FTR is the factor that controls the resolution of the spectrometer. The maximum optical path difference OPD of an interferometer is twice the FTR. Mirrors are moved using actuators and actuators have variations. Hence, the maximum OPD is not constant across the different spectrometers. The differences in maximum OPD will not only introduce differences in resolution and wavelength accuracy, but will also introduce differences in the device line shape function and, hence, change line shape ripples position and amplitude.

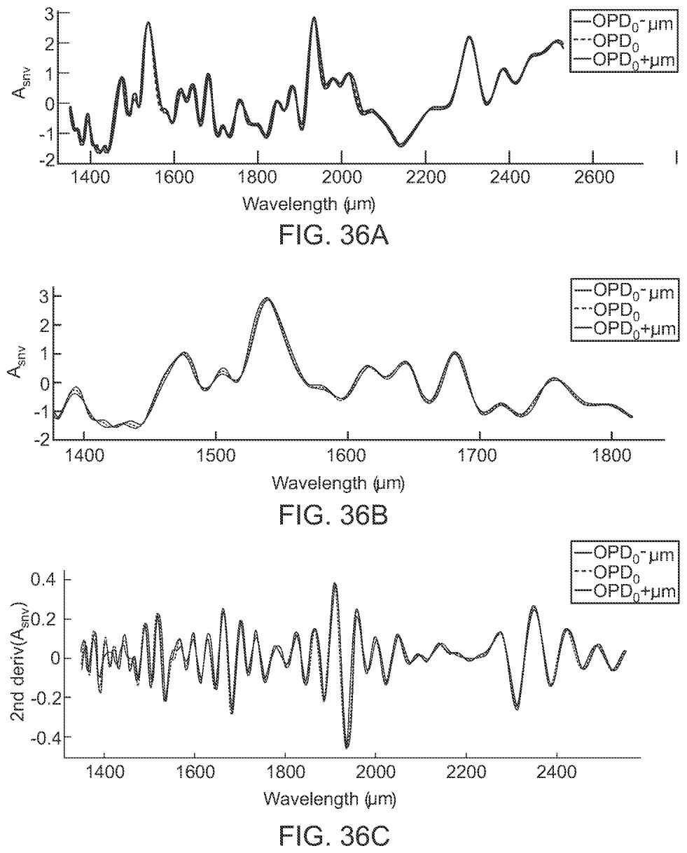

FIGS. 36A-36C illustrate the effect of changing the OPD by ±1 μm on the spectra of a wavelength reference material. Like the self-apodization effect, the spectral converter may further apply an OPD variations effect (e.g., OPD errors) such that a boxcar window W_{BC}(OPD) of variable size can be applied to the measured interferogram as:

I_{new}=Iₒ×W_{BC}(OPD) (25)

Mirror positioning is usually associated with measurement and post processing errors, which lead to OPD error that consequently affects the spectral accuracy. In MEMS FTIR spectrometers, the moveable mirror is driven by a comb drive actuator, where the mirror position is measured by capacitive sensing technique. The capacitance to OPD relation is measured on the production line within the calibration flow of each spectrometer unit. However, there is still a calibration residual error that changes from unit-to-unit. In addition, a residual delay can exist between the measured detector signal versus time and the corresponding capacitance (consequently OPD) signal, which adds to the OPD errors. Such OPD errors can be applied on the measured spectral data to generate artificial spectra that covers these errors and variations from unit-to-unit, such that the artificial units OPD, OPD_{artf}, is a function of the actual unit OPD, OPD_{actual}:

OPD_{artf}=f(OPD_{actual}) (26)

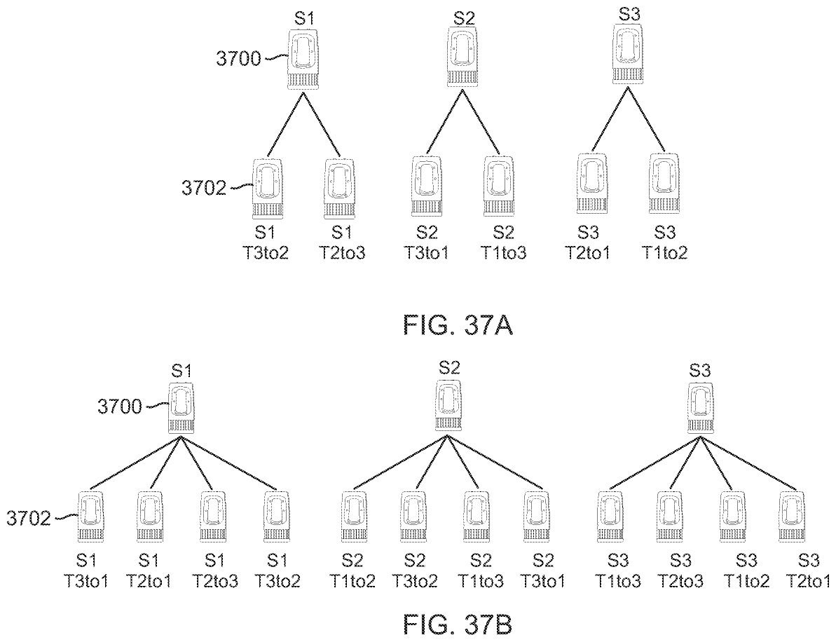

In some examples, generalization can be performed using generalized calibration transfer techniques. FIGS. 37A and 37B illustrate examples of calibration transfer permutation trees according to some aspects. The calibration transfer function can be seen as an error extraction method between different spectral devices. The calibration transfer model extracts the error function between a first spectral device and a second spectral device using a few samples. After that, the error function can be used to transfer any other samples spectra measured by the second spectral device to be similar to samples measured by the first spectral device. Considering that errors between two different spectral devices are not a special case and can be occur with other spectral devices, the extracted transfer function can be applied to other spectral devices to generate new artificial spectral devices. If every two spectral devices are permuted therebetween, they can generate two calibration transfer functions. In this manner, an N number of spectral devices can generate more spectral devices according to the following equation:

N+(N−2)_{N}P₂ (27)

where P is permutation symbol. FIG. 37A illustrates an example of a one-level tree, where six artificial spectral devices 3702 are generated from three original spectral devices. FIG. 37B illustrates an example of an extended one-level tree, where twelve artificial spectral devices 3702 are generated from three original spectral devices 3700.

The generated artificial spectral devices 3702 have a naturalistic instrumental error, not a calculated or emulated error. Moreover, the generated transfer function for different materials and applications can be stored in a library, to be accessible to different users to transfer and generalize their measured samples. The transfer functions can be specific to some materials or they can be generally used to transfer any material measured by a single spectral device to emulate measuring it by many spectral devices. For example, the spectral converter may access the library of pre-calculated stored transfer functions and select which transfer functions should be applied based on the spectral device characteristics.

Adding variations that do not exist to the spectral data used for building chemometrics models may weaken the chemometrics models. Hence, optimization of the generalizer spectral converter parameters for general purposes or for specific applications may be performed. This can be done by integrating the generalizer spectral converter with a chemometrics engine to select the generalizer spectral effects (based on spectral device characteristics), order them according to impact, and tune their parameters according to the chemometric model's performance.

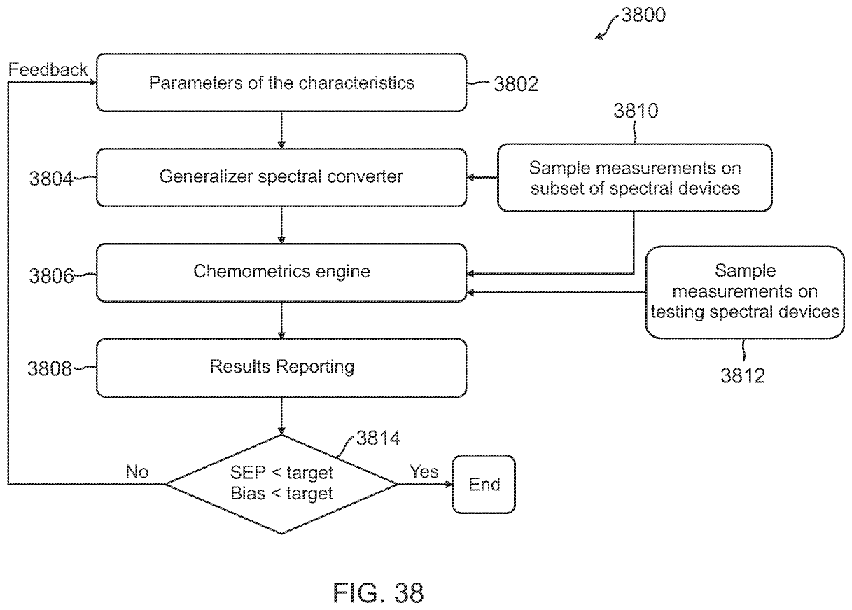

FIG. 38 is an example of a process flow 3800 for optimizing the spectral device characteristics according to some aspects. In the example shown in FIG. 38, parameters of the spectral device characteristics 3802 (e.g., statistical parameters, such as standard deviation and mean values for the different characteristics), along with sample measurements on the subset of spectral devices 3810, may be input to an optimizer function associated with the generalizer spectral converter 3804 for optimization of the parameters. The optimized parameters may then be used to generate of the plurality of artificial spectra. The generated artificial spectra may then be passed to the chemometrics engine 3806, which may then calculate the standard error of prediction (SEP) and their prediction bias (results reporting 3808) based on sample measurements of testing spectral devices 3812. The results 3808 may then be compared against target values at block 3814, and if the results 3808 are less than the target values, the parameters of the characteristics 3802 may be optimized based on the feedback from block 3814. Different parameters may be tested to optimize the prediction performance of different test spectral devices with respect to the subset of spectral devices.

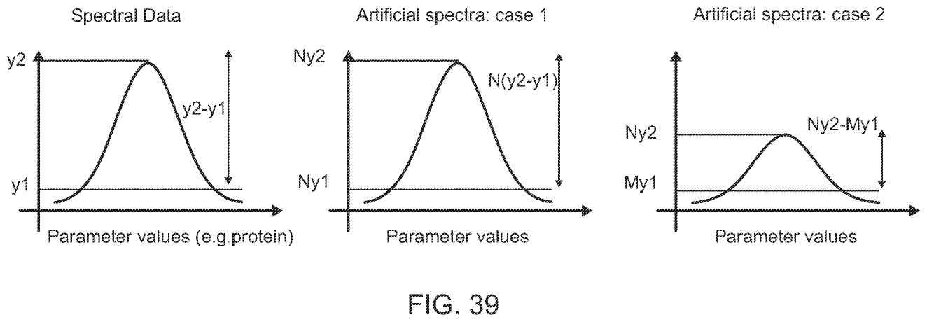

FIG. 39 illustrates an example of alteration of the distribution of the number of spectra versus the value of the analyte in the sample. For example, the histogram on the left shows the distribution of the spectra collected using a sample measurement spectral device (e.g., one of the subset of spectral devices) versus the protein value in a feed sample. The peak of the histogram is y2 at a value of protein x2 in the center of the protein range, while the minimum of the histogram is y1 at the extremes of the protein ranges. If N virtual spectral devices are intended to be produced by the spectral converter, then for each spectrum measured on the sample measurement spectral device, N spectra are produced. Therefore, the new histogram will have a of N multiplied by y2 while the minimum of the histogram is N multiplied by y1, as shown in the middle histogram. Therefore, the difference between the maximum and minimum is magnified by a factor of N. This may cause biasing of the chemometrics model towards improving the accuracy of prediction around x2 values of the sample, while degrading the prediction towards the extremes of the protein range. Therefore, the data augmentation by the spectral converter can take into account such effects. For example, the spectra coming from the subset of spectral devices corresponding to the peak of the histogram can be augmented by a factor of N repressing N remaining spectral devices of the plurality of spectral devices on the production line while the spectra coming from the subset of spectral devices corresponding to the edge of the histogram can be augmented by a factor of M repressing M remaining spectral devices, where M is larger than N, as shown in the histogram on the right.

In some examples, the chemometrics model calibration may undergo several steps of adjustments and refinements to optimize the model performance across all different situations. The chemometrics engine can include different stages to build the model, including selection of the subset of spectral devices, wavelengths folds selection, data unification, model calibration, choosing am optimum number of latent variables, and model adjustment. Initially, the chemometrics engine can be configured to choose the subset of spectral devices used for spectral data collection, and due to the nature of the applications based on miniaturized spectrometers, this stage can be conducted with a precise methodology to ensure optimized performance. Afterward, the chemometric engine can apply the second stage, which is concerned mainly with selecting the best wavelengths ranges and discarding others according to their correlation with the model's dependent variable. In the third stage, the collected data can be expressed to a unification process to remove any undesired fluctuations which may be introduced from improper material measurement or spectral device variations themselves. Finally, in the fourth stage, model calibration is performed, in which the chemometrics model is trained using the optimized dataset to generate the multivariate model. The fifth stage is an optional stage that adjusts the trained model to be able to work with some deviated spectral devices without the need to retrain the model from scratch again.

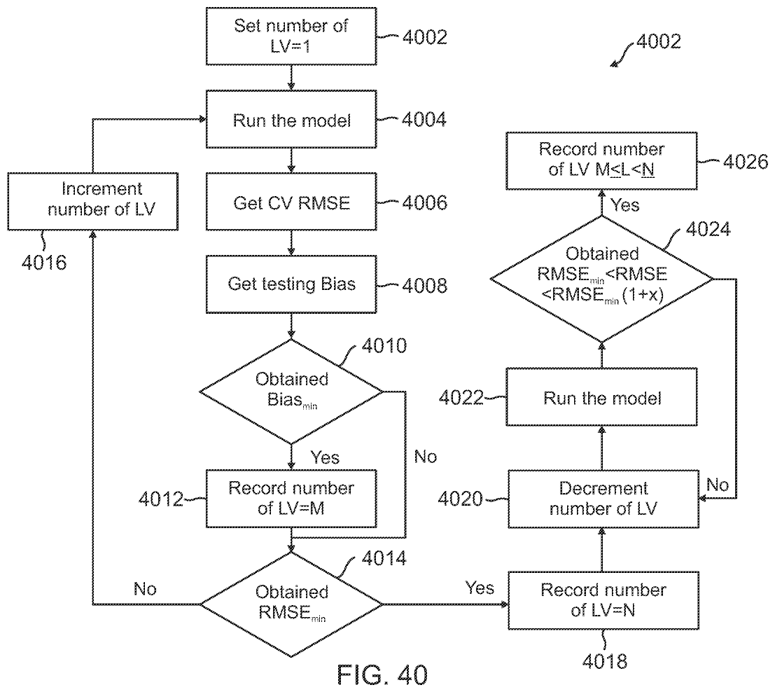

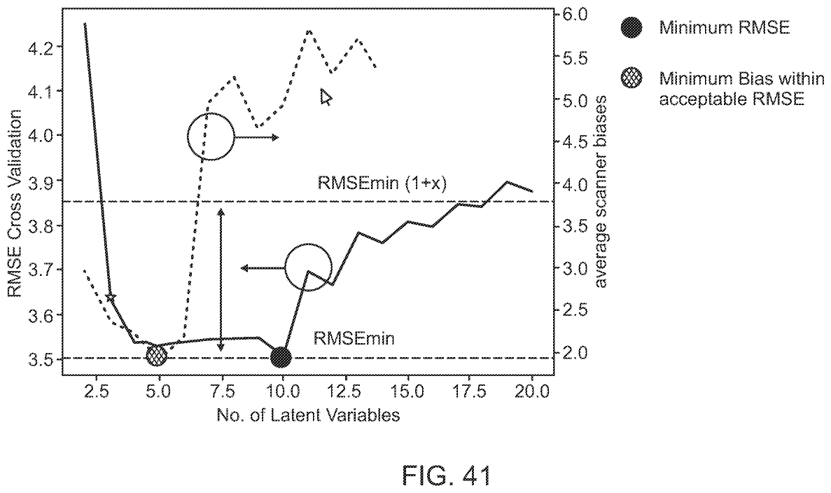

FIG. 40 is a process flow illustrating an example of optimization of the chemometrics model according to some aspects. In the example shown in FIG. 40, optimization is based on the selection of the number of latent variables related to regression algorithms used to produce the chemometrics model. For example, at block 4002, the number of latent variables is set to an initial value, for example 1. At block 4004, the model is applied, and at block 4006, the cost function is calculated, for example the cross validation (CV) RMSE. At block 4008, the bias is also calculated by evaluating the model on remaining (test) spectral devices of the plurality of spectral devices. The bias is then compared to the previous biases obtained during the process, and at block 4010, if the minimum is obtained, at block 4012, the number of latent variable is recorded as M. At block 4014, the RMSE is then compared to the previous RMSE obtained during the process. If the min RMSE is not obtained (No branch of block 4014), then the number of latent variables is incremented at block 4016, and the process is repeated at block 4004. If the min RMSE is reached (Yes branch of block 4014), then at block 4018, the number of latent variables is recorded as N. At block 4020, the number of latent variables is then decremented, and at block 4022, the model is run. Blocks 4020 and 4022 are repeated at block 4024 until the RMSE lies between the minimum RMSE and the minimum RMSE multiplied by a certain factor (1+x), where x is an acceptance criteria. Then, at block 4026, the final number of latent variables L is recorded and used, where the final number is between M and N.

FIG. 41 is a diagram illustrating an example execution of the process shown in FIG. 40. The process 4000 can be applied for optimizing any of the model parameters and the selection of the pre-processing method.

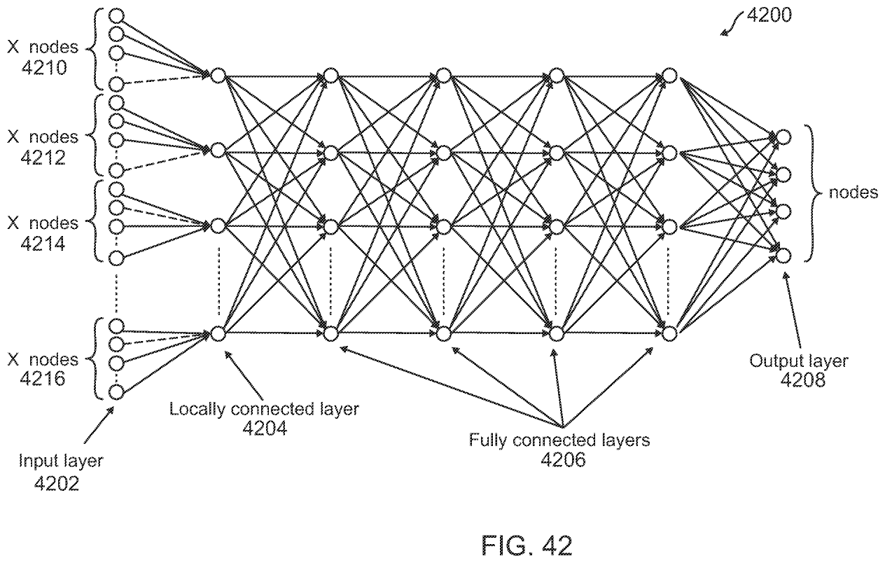

FIG. 42 is a diagram illustrating an example of a neural network according to some aspects. Neural network models are more sophisticated AI algorithms, and may be used in cases having a large number of input features (variables) where better classification performance is expected to be achieved in case of feeding the network with large data set with multiple input features. The neural network system may include multiple layers 4202, 4204, 4206, and 4208. For example, a first layer (input layer) 4202 may contain the inputs (e.g., X Nodes 4210, 4212, 4214, and 4216) and a final layer (output layer) 4208 may contain the values of the calculated hypothesis function h_{θ} (x). The layers between the first and final layer are called the hidden layers with each layer including a number of neurons, and the number of hidden layers may change from one model to another. For example, the hidden layers may include locally connected layers 4204 and fully connected layers 4206. In some examples, the process 4000 shown in FIG. 40 may be utilized to optimize the selection of X for the number of nodes that are mapped to a node in the locally connected layer 4204 of the neural network.

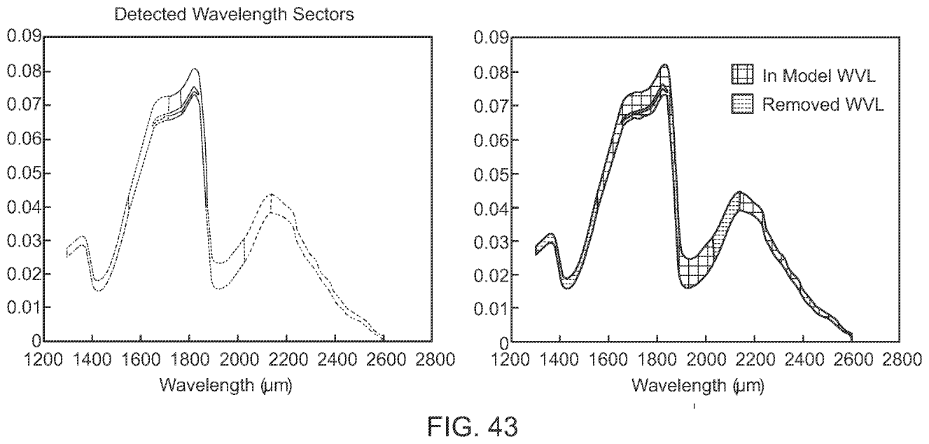

FIG. 43 illustrates an example of optimizing the chemometrics model by applying wavelength (or x-axis in general) selection according to some aspects. In this example, the regions that have minimal contribution to the useful information can be rejected, while leading to less generalization of the model due to unit-to-unit variation. Since the chemical functional groups of materials absorption is represented in short continuous wavelength intervals, an interval-based algorithm may be used to apply the wavelength selection. The selection of wavelength sectors may not be based on a fixed window size, but rather on the different absorption folds found in the spectral data. A preparatory step can locate sectors of wavelengths that have either a peak or a valley, and thus represent part of the absorption signature of the measured material. Later, a selection algorithm can be used in order to discard irrelevant wavelengths sectors from the spectra, as can be seen in the graph on the right in FIG. 43.

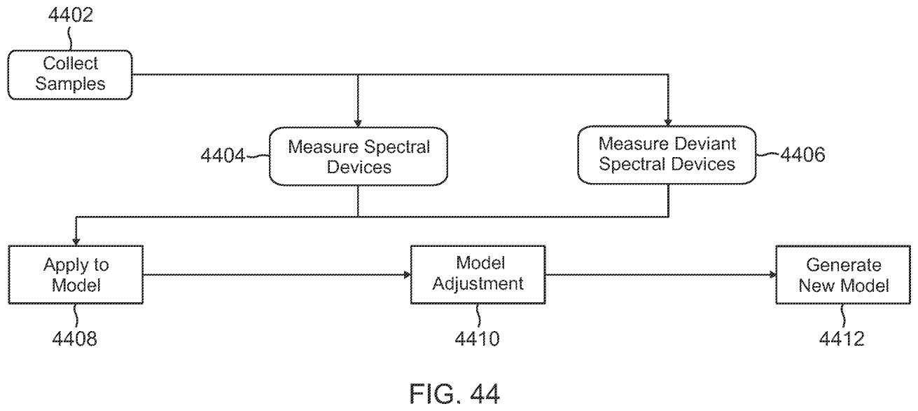

FIG. 44 is a diagram illustraing an example of a spectral modeling adjustment process 4400 for applying model adjustment to further generalize the chemometrics model according to some aspects. In some examples, the process 4400 can be optionally applied to the chemometrics model after the model is completely finalized. The purpose of the model adjustment process 4400 is to enable the chemometrics engine to include some spectral devices (e.g., deviant spectral devices) whose performance deviated significantly from the acceptable range. A deviant spectral device can occur due to various reasons, such as using the spectral device in conditions completely different than the conditions of the model calibration. The model adjustment process 4400 shown in FIG. 44 focuses mainly on including the deviant spectral devices without the need to repeat the whole calibration process and further minimizing any additional measurements required. The process 4400 begins by collecting samples for the adjustment process at block 4402. The number of the adjustment samples may be much smaller than the calibration dataset used to initially produce the chemometrics model. However, samples may be chosen to cover the whole range of the calibration dataset.

At blocks 4404 and 4406, the samples are measured using both the deviant spectral devices and one or more regular sensors (e.g., spectral devices with acceptable performance). At blocks 4408 and 4410, the output for both regular and deviant spectral devices is applied to the chemometrics model and aligned together, such that a new model limited to deviant spectral devices is generated at block 4412.

Mathematically, the model can be adjusted at block 4410 by applying a polynomial correction function as represented in:

Y_{adj}=aₙY_{D}ⁿ+aₙ₋₁Y_{D}ⁿ⁻¹+ . . . +a₀ (28)

where Y_{adj} is the adjusted output of the model, while Y_D and a are the deviated output of the model and the polynomial coefficient respectively.

The coefficients of the polynomial function are optimized based on the readings of the adjustment samples measured at blocks 4404 and 4406, and this can be achieved by applying the following equation:

e=Σ[Y_{R}−(aₙY_{D}ⁿ+aₙ₋₁Y_{D}ⁿ⁻¹+ . . . +a₀)])² (29)

where e is error term which need to be minimized, and Y_{R} is the output of the model of the adjustment samples measured by the regular spectral devices at block 4404.

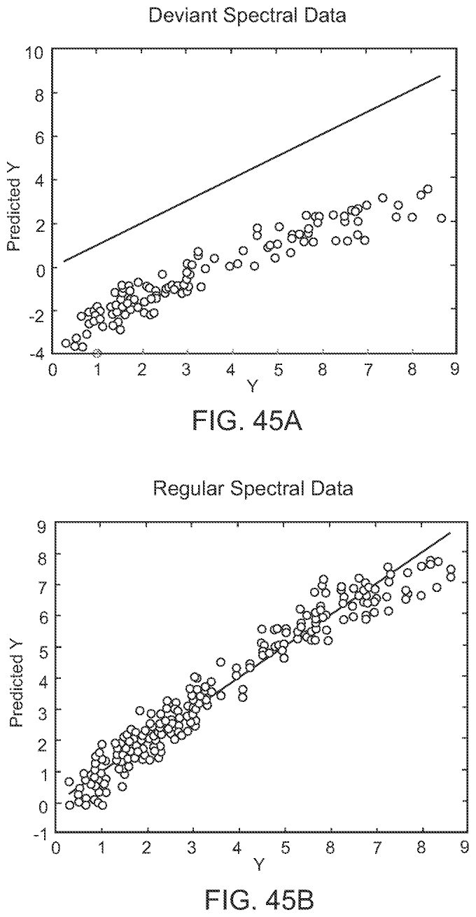

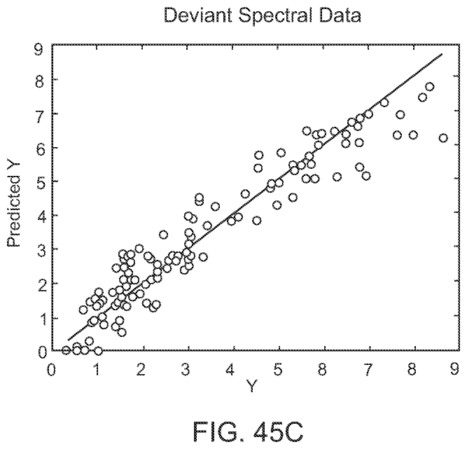

The degree of polynomial correction function can be determined based on the type of the deviation; accordingly, the size of the adjustment samples used for optimizing the polynomial is calculated. However, in most cases, a first-order polynomial may fix the deviations in the output similar to the example shown in FIG. 45A, which can be adjusted using the regular spectral data shown in FIG. 45B to produce a result of the adjusted model illustrated in FIG. 45C.

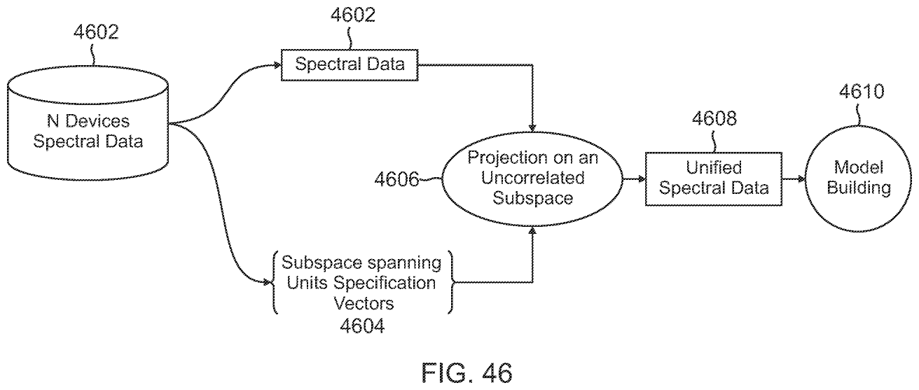

In examples in which there are multiple spectral devices in the subset of spectral devices used to obtain the spectral data, the spectral dataset formed from the multiple spectral devices may be used to feed a multivariate chemometrics model to predict a target property. Two algorithms are discussed herein aimed at unifying the spectral data coming from various spectral devices and obtaining a standardized form of the spectral dataset to be fed to the model building stage. In the first algorithm, shown in FIG. 46, a subspace 4604 spanning the different spectral devices specifications may formed. The spectral data 4602 obtained by N spectral devices (the subset of spectral devices forming a production line) can then be projected on a space 4606 uncorrelated to the specifications subspace 4604. This can be performed by finding the spectral data projection in the orthogonal complement direction the specifications subspace. Let A be the subspace spanning the multiple device system specifications vectors. Then, another space B can be found such that B=A_{⊥}; the orthogonal complement subspace of A as follows:

B=A⊥={\vec{x}ε [symbol] ⁿ|\vec{x}.\vec{a}=0∀\vec{a}εA} (30)

Then, a unified spectral dataset 4608 can be found by projecting the multi-device spectral data S on subspace B, generating SB represented in an uncorrelated subspace to A. The unified spectral data 4608 may then be used to build the chemometrics model 4610.

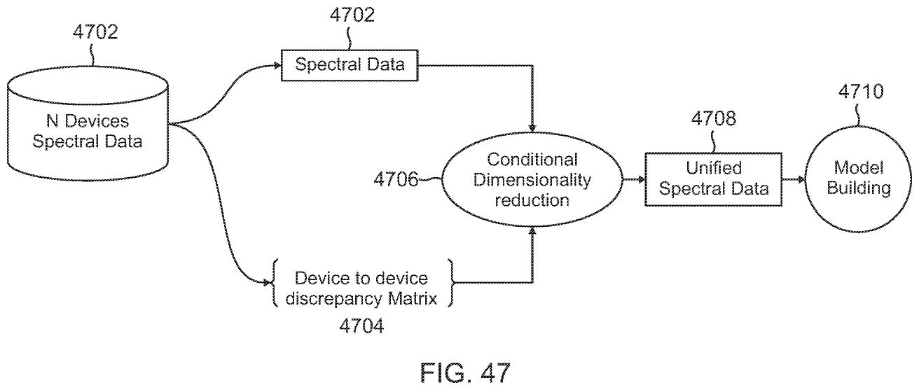

The second algorithm, shown in FIG. 47 is an algorithm that minimalizes the per measurement discrepancies among different spectral devices through an optimization problem. In this algorithm, a matrix 4704 is formed describing the device-to-device discrepancies for each measurement in the spectral dataset 4702. Regularized dimensionality reduction 4706 of the multi-device spectral dataset can then be applied. The device-to-device discrepancy matrix (D) 4704 of the multi-device spectral dataset can be found by subtracting each spectral device's measured sample spectrum by the mean of all spectral device measurements of the same sample as in:

D=Matrix[Xⱼ(i)−X̅ⱼ̅] (31)

where Xⱼ(i) s the spectrum of sample j for device i and X̅ⱼ̅ is the mean of spectra of sample j for all devices. Any method of regularized components reductions can be used such as in:

min∥X−(X P)PT∥+μ∥D∥S.T.P PT=1 (32)

where X is the spectral data, P and T are the principle components loadings and scores, and μ is the regularization rate.

In some examples, it may not be practical to measured real samples; such as food, feed, soil and others on the production line, since the properties of the samples change with time and usage. At the same time, there is a need to test the spectral devices after calibration and ensure that the bias/slope of the chemometrics models applied to the calibrated spectral devices are within the accepted range. If not, the spectral devices may need to be re-calibrated.

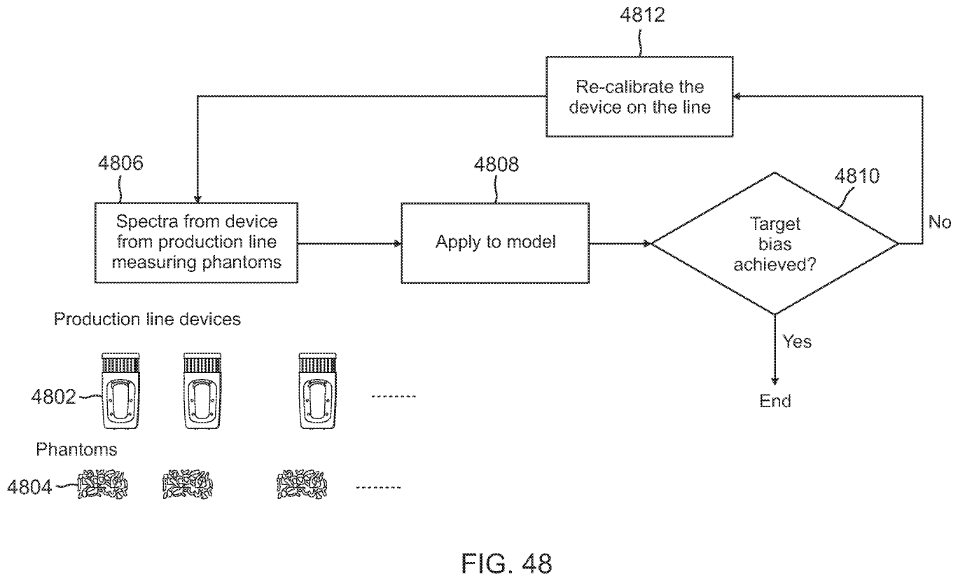

FIG. 48 is a process flow illustrating an example process for testing spectral devices according to some aspects. In the example shown in FIG. 48, a plurality of production line devices 4802 may be tested using phantom samples 4804. The phantom samples 4804 can be made of stable substances that do not change with time and that are easy to preserve. The phantom samples 4804 are made such that their absorbance spectra are near or the same as the real samples' spectra. For example, the absorbance peaks strength and wavelength location may be at approximately the same values as the real samples. Several phantom samples composed of different concentrations may first measured on the subset of spectral devices and a chemometrics model can be built with the generalizer spectral converter. This process may be performed once or it may be repeated from time to time, for example on monthly basis. The chemometrics model may then be used to qualify the produced devices, as shown in FIG. 48.

As shown in FIG. 48, the phantom samples 4804 are measured on the newly produced production line spectral devices 4802. For each new spectral device, the resulting spectra 4806 from the measured phantom samples are fed to the chemometrics model at block 4808 and the prediction results are recorded. The results are compared with the calibration results and the bias/slope are calculated. If the target is achieved at block 4810, the spectral device passes the qualification on the production line. If not, at block 4812, the spectral device is re-calibrated and the process is repeated until the target is achieved.

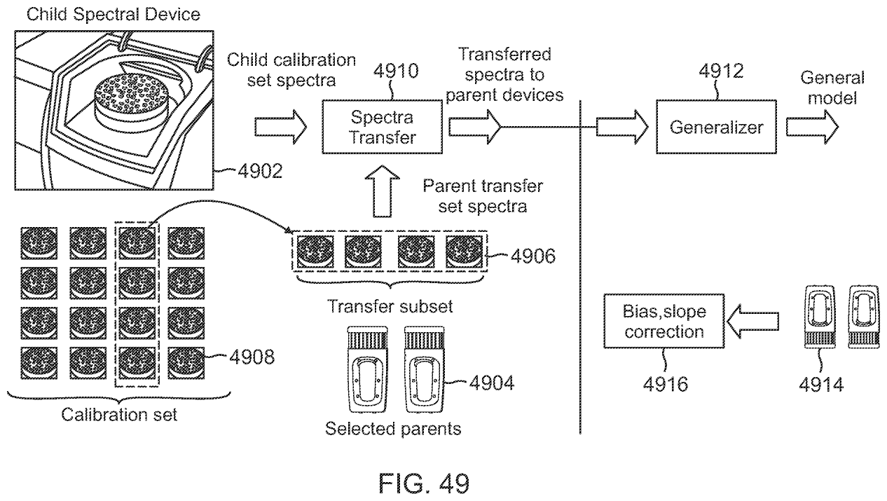

The generalization can be also extended to a globalization process as shown in FIG. 49. In the example shown in FIG. 49, a calibration database that was built previously for a spectral device that resembles a different spectrometer product is used. For example, the different spectrometer can be a references bench top device or a device based on another method of analyzing the spectrum, for example such as a diffraction grating spectrometer, while the spectral devices are based on the FTIR. First, a calibration transfer can be carried out from the child (reference) device 4902 to one or more parent devices 4904 (e.g., selected spectral devices of the plurality of spectral devices for which the calibration model was originally generated). This can be based on one of various transfer methods using a transfer subset of samples 4906 of the original calibration set of samples 4908. The transfer set 4907 is measured on the child device 4902 and the parents 4904. Then, at block 4910, mathematical operations are used to transfer the calibration database spectra from the child to the parents. At block 4912, a generalizer operation is performed to generalize the chemometrics model built on the parent devices to all the production line devices (child devices). This can be based on any of the schemes described previously, for example, using a statistical spectral converter then running a chemometrics model. The success of the whole process may be qualified by testing some samples on testing child devices 4914 and checking the prediction correlation, SEP and bias at block 4916. In the case of unacceptable results, the parameters of the statistical converter may be tuned and the process is repeated. This is to account for the fact that the original spectra is coming from a child device that is completely different from the parent spectral devices. In some examples, premium testing devices 4914 can be chosen and a process of bias/slop correction for obtaining higher accuracy results may be selected.

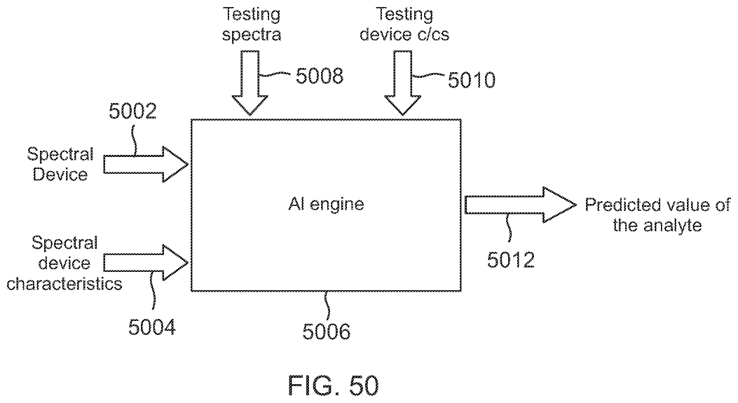

In some examples, testing of child spectral devices may be performed as indicated in the example shown in FIG. 50. In FIG. 50, the original spectral data 5002 from the parent devices, along with the spectral device characteristics 5004 of the parent devices may be fed to the artificial intelligence (AI) engine 5006 to build the calibration (chemometrics model). In addition, the testing device spectral data 5008, along with the testing device characteristics 5010 may be fed to the AI engine 5006 for use predicting the value of analyte 5012. Different device characteristics can be used from the production line data as features input to the AI engine 5006. This includes, but is not limited to, background material power spectral density, reflection or absorption spectrum of a reference material, such as polystyrene or certificated reference materials, reflection or absorption spectrum of a material with sharp lines, such as Talc or rare earth oxides, measured spectra without having a sample (e.g., back reflection), SNR verses wavelength, and so on.

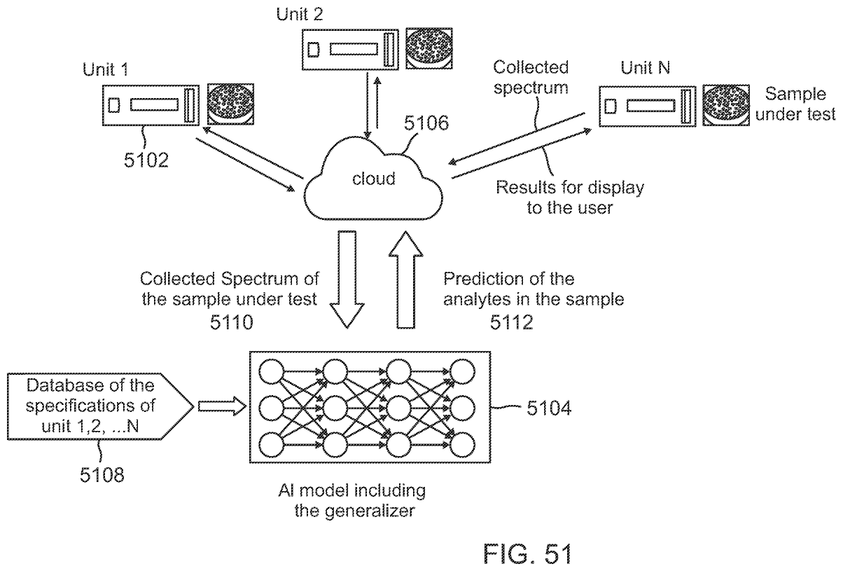

FIG. 51 is a diagram illustrating a cloud-based spectral modeling system according to some aspects. The cloud-based spectral modeling system enables easier access to cloud-based AI engines/chemometrics models and optimizes the needed memory to save the specifications of all the produced units 5102 (e.g., Unit 1, Unit 2, . . . , Unit N). A generalized artificial intelligence model 5104 can be stored on a local server, external on the cloud 5106 (as shown in FIG. 51), on memory of the Unit or on a mobile app, for example. In the case of local storage, a database 5108 of the specifications of the Units 5102 is locally connected to the model 5104. In the case of external storage, the connection between the database of the specifications 5108 and the model 5104 will be through the cloud 5106. In an example, the chemometrics model may be stored on the cloud 5106 and the Units 5102 may access the chemometrics model on the cloud or download the chemometrics mode to be used locally on the Unit or a controlling device (e.g., cell phone, computer, etc.) controlling the Unit. Given the unit ID of the Unit 5102 being tested, the model 5104 can recall the specification of the unit and use it to make a prediction 5112 together with the collected spectrum 5110 of the sample using the unit.

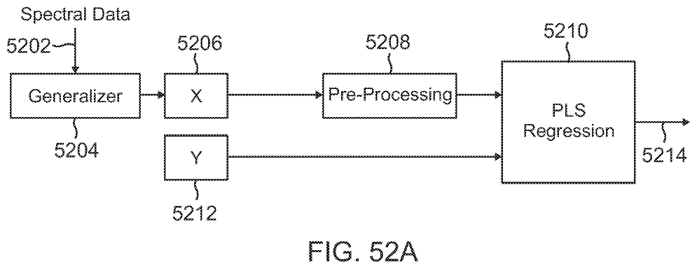

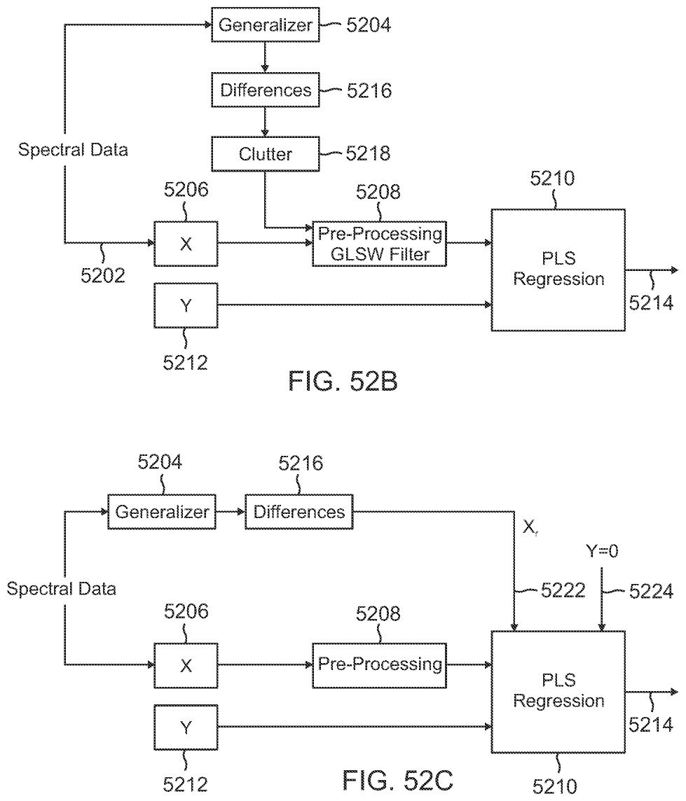

FIGS. 52A-52C are diagrams illustrating examples of generalized chemometrics model building using a generalizer spectral converter according to some aspects. In the example shown in FIG. 52A, a generalizer spectral converter 5204 is fed the spectral data 5202 measured by a subset of the plurality of spectral devices to output a spectral data matrix (represented by box X) 5206. The spectral data matrix 5206 shown in FIG. 52A corresponds to a plurality of artificial spectra that covers different spectral device variations. Pre-processing 5208 and a PLS regression 5210 may be performed on the artificial spectra using reference values 5212 for the material parameters for the different training samples (e.g., protein/moisture content) (represented by box Y) to produce a chemometrics model 5214 that is robust against these variations.

In the example shown in FIG. 52B, the spectral data 5202 obtained by the subset of spectral devices forms the spectral data matrix 5206 that is input to the pre-processing block 5208. In addition, a generalizer spectral converter 5204 is fed the spectral data 5202, along with spectral device characteristics (e.g., which may be obtained from spectra of production line reference materials, such as Poly, MRC, Talc, etc.) to produce a plurality of artificial spectra. A differences matrix 5216 may be formed by extracting, for each sample spectrum, a difference spectra from the difference between the generated spectra by the generalizer 5204 and the original sample spectrum. The differences matrix corresponds to clutter signals 5218 indicative of device variations. The device variations can be compensated using a Generalized Least Square Weighting (GLSW) filter in the pre-processing block 5208 that is built/trained to remove the clutter signals that correspond to device variations in the artificial spectra and the original spectral data to produce filtered data. The output of the pre-processing block 5208, along with the reference values 5212, may then be input to the PLS regression block 5210 to produce the chemometrics model 5214.

In the example shown in FIG. 52C, the spectral data 5202 obtained by the subset of spectral devices forms the spectral data matrix 5206 that is input to the pre-processing block 5208. In addition, a generalizer spectral converter 5204 is fed the spectral data 5202 to produce a plurality of artificial spectra. A differences matrix 5216 may be formed by extracting, for each sample spectrum, a difference spectra from the difference between the generated spectra by the generalizer 5204 and the original sample spectrum corresponding to errors added by device variations to produce a repeatability file. The chemometrics model 5214 is trained by the PLS regression block 5210 using the repeatability file with corresponding Y (reference) values=0, such that the model acts to minimize the effect of the device variations.

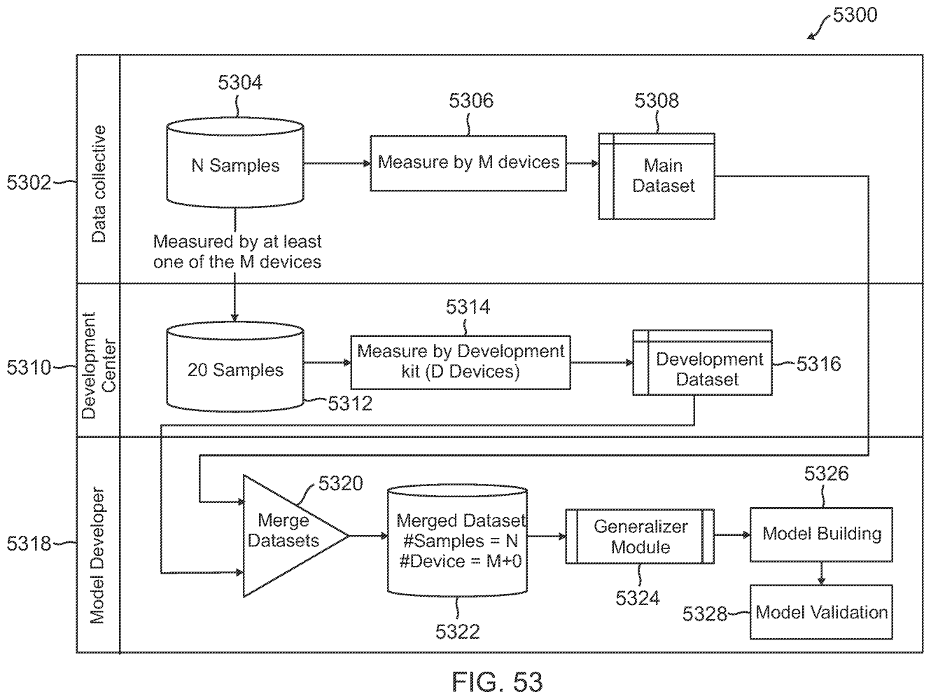

FIG. 53 is a diagram illustrating another example of a spectral modeling system 5300 according to some aspects. The system 5300 shown in FIG. 53 is configured to build robust, spectral device independent models with a generalized performance across any spectral device. The system 5300 is formed of three stages, data collection 5302, data augmentation performed by development centers 5310, and finally model development 5318.

In the data collection stage 5302 that is performed on the model developer side, a number of samples (N) 5304 of varying conditions and constituents' concentrations are collected and prepared for measurement. Spectral measurements of the N samples are performed at block 5306 using M spectral devices (the main kit) corresponding to a subset of the plurality of spectral devices to produce a main spectral dataset 5308. Each of the M spectral devices measures a different set of samples. Each spectral device measures a set of samples with high distribution across the range of the parameters of interest (for example, samples with varying protein values across the full range of protein for this material). The samples are referenced using wet chemistry or NIR benchtop devices. The sample condition should be maintained during spectral measurement on the spectral devices and during referencing to ensure consistency. A subset of the samples (e.g., 20 samples) 5312 are further measured by at least one of the M spectral devices and preferably measured by multiple of the M spectral devices for extra data augmentation. The subset of samples 5312 shall be preserved and sealed carefully and shipped to the closest development center to developer/customer location. References for these samples shall be provided.

Development centers are dedicated for model augmentation, validation and maintenance. In the development center stage 5310, the subset of samples 5312 received from the model developer is measured by a development kit composed of a larger number of spectral devices (D spectral devices, where D>M and D<T, where T it the total number of spectral devices) to cover more regions on the space of variations. The generated dataset is referred to as the development dataset 5316 and is used to augment the main dataset 5308 to introduce more data to the built model.

In the model developer stage 5318, the main dataset 5308 and the development dataset 5316 are merged at block 5320 forming a high coverage dataset 5322. The merged dataset 5322 is used to generate an initial model to check for any outlier readings. The cleaned dataset is fed to a generalizer module 5324. The generalizer module 5324 generates a plurality of artificial spectra from the cleaned dataset and spectral device characteristics of the production line (T) spectral devices. The artificial spectra represent virtual spectral devices that map to the production line distribution of spectral devices. The output of the generalizer module 5324 is an augmented dataset containing the merged dataset and artificial data. Model developing is performed at block 5326 on the final generalized dataset resulting in a scalable model that performs uniformly on any new spectral device. In some examples, spectral device out cross validation may be performed at block 5328 during model building to optimize the model parameters such that they best perform on new spectral devices.

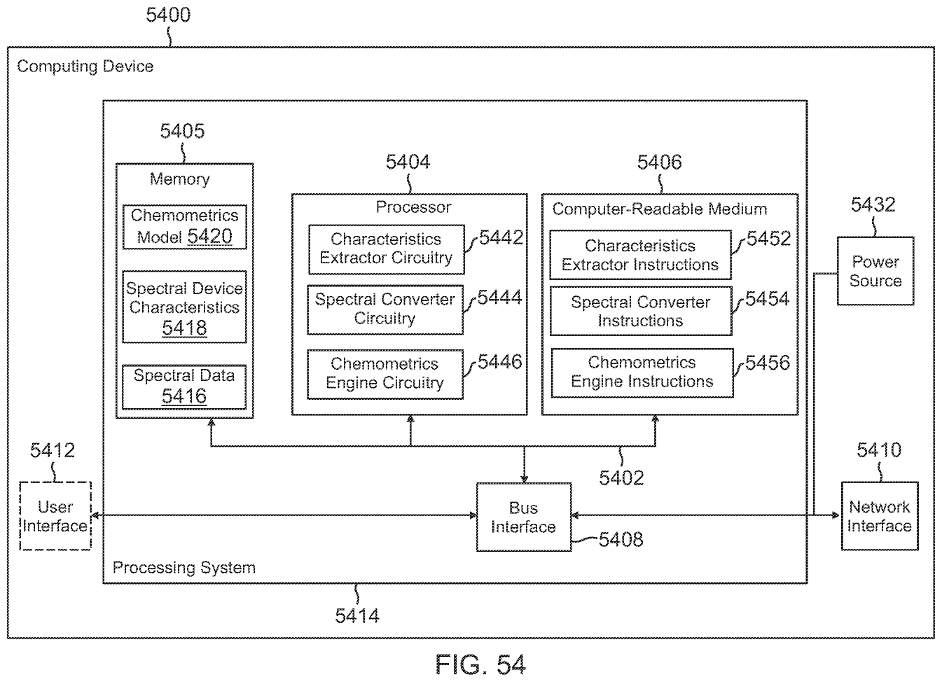

FIG. 54 is a block diagram illustrating an example of a hardware implementation for a computing device 5400 employing a processing system 5414 according to some aspects. For example, the computing device 5400 may correspond to a personal computer, server, handheld device (e.g., cell phone or tablet), cloud-based device, or any other suitable computing device.

The computing device 5400 may be implemented with a processing system 5414 that includes one or more processors 5404. Examples of processors 5404 include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. In various examples, the computing device 5400 may be configured to perform any one or more of the functions described herein. That is, the processor 5404, as utilized in the computing device 5400, may be used to implement any one or more of the processes and procedures described herein. In some examples, the processing system 5414 may be distributed among various entities, which may be coupled via a direct or indirect connection (e.g., wired or wireless).

The processor 5404 may in some instances be implemented via a baseband or modem chip and in other implementations, the processor 5404 may include a number of devices distinct and different from a baseband or modem chip (e.g., in such scenarios as may work in concert to achieve examples discussed herein). And as mentioned above, various hardware arrangements and components outside of a baseband modem processor can be used in implementations, including RF-chains, power amplifiers, modulators, buffers, interleavers, adders/summers, etc.

In this example, the processing system 5414 may be implemented with a bus architecture, represented generally by the bus 5402. The bus 5402 may include any number of interconnecting buses and bridges depending on the specific application of the processing system 5414 and the overall design constraints. The bus 5402 links together various circuits including one or more processors (represented generally by the processor 5404), a memory 5405, and computer-readable media (represented generally by the computer-readable medium 5406). The bus 5402 may also link various other circuits such as timing sources, peripherals, voltage regulators, and power management circuits, which are well known in the art, and therefore, will not be described any further.

A bus interface 5408 provides an interface between the bus 5402, a network interface 5410, and a power source 5432. The network interface 5410 provides a means for communicating with various other apparatus over a transmission medium (e.g., wireline or wireless) The power source 5432 provides a means for supplying power to various components in the computing device 5400. Depending upon the nature of the apparatus, a user interface 5412 (e.g., keypad, display, touch screen, speaker, microphone, control knobs, etc.) may also be provided. Of course, such a user interface 5412 is optional, and may be omitted in some examples.

The processor 5404 is responsible for managing the bus 5402 and general processing, including the execution of software stored on the computer-readable medium 5406. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software, when executed by the processor 5404, causes the processing system 5414 to perform the various functions described below for any particular apparatus. The computer-readable medium 5406 and the memory 5405 may also be used for storing data that is utilized by the processor 5404 when executing software. For example, the memory 5405 may store one or more of spectral data 5416, spectral device characteristics 5418, and/or a chemometrics model 5420.

The computer-readable medium 5406 may be a non-transitory computer-readable medium. A non-transitory computer-readable medium includes, by way of example, a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium 5406 may reside in the processing system 5414, external to the processing system 5414, or distributed across multiple entities including the processing system 5414. The computer-readable medium 5406 may be embodied in a computer program product. By way of example, a computer program product may include a computer-readable medium in packaging materials. In some examples, the computer-readable medium 5406 may be part of the memory 5405. Those skilled in the art will recognize how best to implement the described functionality presented throughout this disclosure depending on the particular application and the overall design constraints imposed on the overall system.

In some aspects of the disclosure, the processor 5404 may include circuitry configured for various functions. For example, the processor 5404 may include characteristics extractor circuitry 5442, configured to generate spectral device characteristics 5418 representing spectral variations in a plurality of spectral devices (e.g., that form a production line). In some examples, the spectral device characteristics 5418 may include at least one of signal-to-noise ratio (SNR), wavelength repeatability, wavelength error, absorbance scaling, self-apodization function, baseline shift, back reflection, thermal drift, environmental drift, optical path difference (OPD) variation, or Etalon effect.

In some examples, the characteristics extractor circuitry 5442 may be configured to receive background spectra from at least one spectral device using a reference tile or transmission sampling accessory and to extract the SNR based on the background spectra. In some examples, the characteristics extractor circuitry 5442 may be configured to receive measured spectra from at least one spectral device measured using a wavelength reference material and to extract at least one of the wavelength repeatability or the wavelength error based on the measured spectra. In some examples, the characteristics extractor circuitry 5442 may be configured to receive at least one interferogram from at least one spectral device measured using a narrowband optical filter and to extract the self-apodization function based on the at least one interferogram. In some examples, the characteristics extractor circuitry 5442 may be configured to receive measured spectra from at least one spectral device of the remaining spectral devices measured with variable temperature and to extract the thermal drift based on the measured spectra.

In some examples, the characteristics extractor circuitry 5442 may be configured to receive measured spectra of universal samples different than the plurality of samples from at least a portion of the plurality of spectral devices and to extract the spectral device characteristics of the plurality of spectral devices using measured spectra. In some examples, the portion includes all of the plurality of spectral devices. In other examples, the portion includes selected spectral devices of the plurality of spectral devices having corresponding spectral device characteristics covering a space of variations including corners of production line characteristics of the production line.

In some examples, the characteristics extractor circuitry 5442 may be configured to generate the spectral device characteristics 5418 based on statistical information related to the production line. For example, the statistical information may include various statistical parameters, such as the mean value, standard deviation, skewness, or kurtosis, and a probability distribution (histogram) of each of the statistical parameters. The characteristics extractor circuitry 5442 may further be configured to execute characteristics extractor instructions (software) 5452 stored in the computer-readable medium 5406 to implement one or more of the functions described herein.

The processor 5404 may further include spectral converter circuitry 5444, configured to receive spectral data 5416 of a plurality of samples from a subset of a plurality of spectral devices and to further receive the spectral device characteristics 5418 representing spectral variations in the plurality of spectral devices. The spectral converter circuitry 5444 may further be configured to generate a plurality of artificial spectra representing remaining spectral devices of the plurality of spectral devices based on the spectral data 5416 and the spectral device characteristics 5418. In some examples, the spectral data 5416 includes measurements of phantom samples corresponding to the plurality of samples, where each of the phantom samples includes a stable substance having a same absorbance spectra as one of the one or more samples.

In some examples, the spectral converter circuitry 5444 may be configured to apply a spectral variance function to the spectral data to produce processed spectral data representative of variances in the subset of the plurality of spectral devices. In some examples, the spectral converter circuitry 5444 may be configured to apply a spectral correction function to the spectral data to produce processed spectral data that removes uncontrolled variances in the subset of the plurality of spectral devices. In some examples, the spectral converter circuitry 5444 may be configured to apply a spectral modulation and perturbation function to the spectral data to produce processed spectral data spanning different levels of aging and environmental conditions variations. In some examples, the spectral converter circuitry 5444 may be configured to apply an optical head variance function to the spectral data to produce processed spectral data that accounts for different optical head configurations in the subset of the plurality of spectral devices.

In some examples, the spectral converter circuitry 5444 may be configured to apply a set of apodization functions to the spectral data (or the processed spectral data) to produce the plurality of artificial spectra. In some examples, the spectral converter circuitry 5444 may be configured to add wavelength errors to the spectral data (or the processed spectral data) to produce the plurality of artificial data. In some examples, the spectral converter circuitry 5444 may be configured to add noise across a spectral range corresponding to a signal-to-noise ratio (SNR) distribution to the spectral data (or the processed spectral data) to produce the plurality of artificial spectra. In some examples, the spectral converter circuitry 5444 may be configured to scale an absorbance spectrum of the spectral data (or the processed spectral data) using a wavelength dependent scaling factor to produce the plurality of artificial spectra. In some examples, the spectral converter circuitry 5444 may be configured to multiply the spectral data (or processed spectral data) by a thermal drift factor across wavelength to produce the plurality of artificial spectra.

In some examples, the spectral converter circuitry 5444 may be configured to add baseline variations to absorbance of the spectral data (or processed spectral data) to produce the plurality of artificial spectra. In some examples, the spectral converter circuitry 5444 may be configured to add back reflection spectra to the spectral data (or processed spectral data) to produce the plurality of artificial spectra. In some examples, the spectral converter circuitry 5444 may be configured to multiply an Etalon effect to the spectral data (or the processed spectral data) to produce the plurality of artificial spectra. In some examples, the spectral converter circuitry 5444 may be configured to multiply background material reflectance variations associated with background materials used to produce the spectral device characteristics to the spectral data to produce the plurality of artificial spectra. In some examples, the spectral converter circuitry 5444 may be configured to apply optical path difference (OPD) errors to the spectral data to produce the plurality of artificial spectra.

In some examples, the spectral converter circuitry 5444 may be configured to optimize the spectral device characteristics based on measured values from test spectral devices. In some examples, the spectral converter circuitry 5444 may be configured to alter a distribution of the plurality of artificial spectra with respect to a corresponding measured value of the plurality of samples. In some examples, the spectral converter circuitry 5444 may be configured to extract difference spectra between the plurality of artificial spectra and the spectral data, where the difference spectra corresponds to clutter signals indicative of device variations between the plurality of devices. The spectral converter circuitry 5444 may then be configured to filter the clutter signals from the spectral data and the plurality of artificial data to produce processed spectral data used to generate the chemometrics model.

In some examples, the spectral converter circuitry 5444 may be configured to receive a development dataset of a subset of the plurality of samples measured by a development kit including an additional subset of the plurality of spectral devices larger than the subset of the plurality of spectral devices. The spectral converter circuitry 5444 may then be configured to merge the spectral data and the development dataset to produce a merged dataset and to use the merged dataset to generate the plurality of artificial spectra. In some examples, the spectral converter circuitry 5444 may be configured to access a library of pre-calculated stored transfer functions, select one or more selected transfer functions of the pre-calculated stored transfer functions based on the spectral device characteristics, and use the one or more selected transfer functions to generate the artificial spectra. In some examples, the spectral converter circuitry 5444 may further be configured to extract difference spectra between the plurality of artificial spectra and the spectral data, where the difference spectra corresponds to a repeatability file indicative of device variations between the plurality of devices. The spectral converter circuitry 5444 may further be configured to execute spectral converter instructions (software) 5454 stored in the computer-readable medium 5406 to implement one or more of the functions described herein.

The processor 5404 may further include chemometrics engine circuitry 5446 configured to produce a chemometrics model for one or more parameters associated with the plurality of samples based on the spectral data and the plurality of artificial spectra. In some examples, the chemometrics engine circuitry 5446 may be configured to select the subset of the plurality of spectral devices, select one or more wavelength ranges for the spectral data, removing fluctuations in the spectral data resulting from improper measurement or variations in the subset of the plurality of spectral devices, and train the chemometrics model based on the spectral data and the plurality of artificial spectra.

In some examples, the chemometrics engine circuitry 5446 may be configured to adjust the chemometrics model using additional spectral data from deviant spectral devices of the plurality of spectral devices that deviate in performance from regular spectral devices of the plurality of spectral devices. In some examples, the chemometrics engine circuitry 5446 may be configured to optimize a number of latent variables used to produce the chemometrics model to minimize a bias between test spectral devices of the remaining spectral devices and produce a root mean squared error within a specified range from a target minimum value. In some examples, the chemometrics engine circuitry 5446 may be configured to identify a unified spectral dataset for the subset of spectral devices based on the spectral data by projecting the spectral data onto a space that is uncorrelated with a subspace of spectral device specification discrepancies. In some examples, the chemometrics engine circuitry 5446 may be configured to form a matrix describing discrepancies between the subset of the plurality of spectral devices for each measurement in the spectral data and to apply a conditional dimensionality reduction on the sensor data using the matrix.

In some examples, the chemometrics engine circuitry 5446 may be configured to calibrate additional spectral devices using the phantom samples and the chemometrics model. In some examples, the chemometrics engine circuitry 5446 may be configured to generate a transfer function using a set of samples measured on one or more of the plurality of spectral devices and a different spectral device comprising a different configuration than any of the plurality of spectral devices. The chemometrics engine circuitry 5446 may then be configured to generalize the chemometrics model to include the different spectral device based on the transfer function.

In some examples, the chemometrics engine circuitry 5446 may be configured to producing the chemometrics model for the one or more samples based on the spectral data, the plurality of artificial spectra, and additional spectral device characteristics of the subset of the plurality of spectral devices. In some examples, the chemometrics engine circuitry 5446 may be configured to receive a sample measurement of a sample under test from a test spectral device of the plurality of test devices, where the sample under test corresponding to one of the one or more samples, receive test spectral device characteristics of the test spectral device and generate a result using the chemometrics model, the sample measurement, and the test spectral device characteristics.

In some examples, the chemometrics engine circuitry 5446 may be a cloud-based artificial intelligence engine configured to store the chemometrics model and test spectral device characteristics and other test spectral device characteristics of other test spectral devices of the plurality of spectral devices. In some examples, the chemometrics model 5420 may be a cloud-based chemometrics model accessible to the plurality of spectral devices. In some examples, the chemometrics engine circuitry 5446 may be configured to use the repeatability file together with corresponding zero reference values to generate the chemometrics model. The chemometrics engine circuitry 5446 may further be configured to execute chemometrics engine instructions (software) 5456 stored in the computer-readable medium 5406 to implement one or more of the functions described herein.

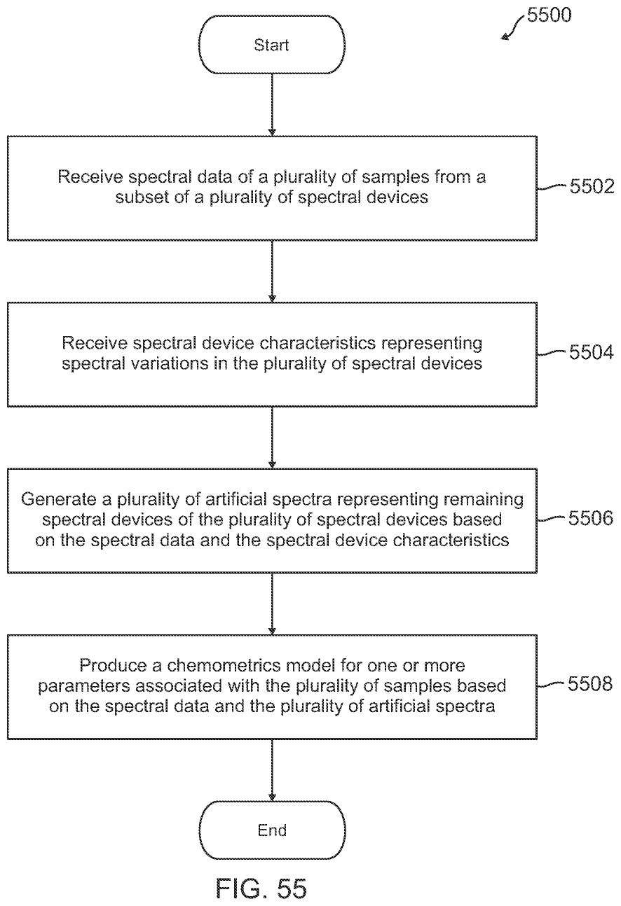

FIG. 55 is a flow chart illustrating an exemplary process 5500 for producing a generalized chemometrics model according to some aspects. As described below, some or all illustrated features may be omitted in a particular implementation within the scope of the present disclosure, and some illustrated features may not be required for implementation of all embodiments. In some examples, the process 5500 may be carried out by the computing device 5400 illustrated in FIG. 54 or a spectral modeling system including one or more computing devices. In some examples, the process 5500 may be carried out by any suitable apparatus or means for carrying out the functions or algorithm described below.

At block 5502, the spectral modeling system may receive spectral data of a plurality of samples from a subset of a plurality of spectral devices. At block 5504, the spectral modeling system may receive spectral device characteristics representing spectral variations in the plurality of spectral devices. At block 5506, the spectral modeling system may generate a plurality of artificial spectra representing remaining spectral devices of the plurality of spectral devices based on the spectral data and the spectral device characteristics. At block 5508, the spectral modeling system may produce a chemometrics model for one or more parameters associated with the plurality of samples based on the spectral data and the plurality of artificial spectra.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another—even if they do not directly physically touch each other. For instance, a first object may be coupled to a second object even though the first object is never directly physically in contact with the second object. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in FIGS. 1-55 may be rearranged and/or combined into a single component, step, feature or function or embodied in several components, steps, or functions. Additional elements, components, steps, and/or functions may also be added without departing from novel features disclosed herein. The apparatus, devices, and/or components illustrated in FIGS. 1-54 may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

What is claimed is:

1. A spectral modeling system, comprising:
a spectral converter configured to receive spectral data of a plurality of samples from a subset of a plurality of spectral devices and spectral device characteristics representing spectral variations in the plurality of spectral devices, the spectral converter further configured to generate a plurality of artificial spectra representing remaining spectral devices of the plurality of spectral devices based on the spectral data and the spectral device characteristics; and
a chemometrics engine configured to produce a chemometrics model for one or more parameters associated with the plurality of samples based on the spectral data and the plurality of artificial spectra.

2. The spectral modeling system of claim 1, wherein the spectral device characteristics comprise at least one of signal-to-noise ratio (SNR), wavelength repeatability, wavelength error, absorbance scaling, self-apodization function, baseline shift, back reflection, thermal drift, environmental drift, optical path difference (OPD) variation, or Etalon effect.

3. The spectral modeling system of claim 2, further comprising:
a characteristics extractor configured to receive background spectra from at least one spectral device of the remaining spectral devices measured using a reference tile or transmission sampling accessory, and extract the SNR based on the background spectra.

4. The spectral modeling system of claim 2, further comprising:
a characteristics extractor configured to receive measured spectra from at least one spectral device of the remaining spectral devices measured using a wavelength reference material and extract at least one of the wavelength repeatability or the wavelength error based on the measured spectra.

5. The spectral modeling system of claim 2, further comprising:
a characteristics extractor configured to receive at least one interferogram from at least one spectral device of the remaining spectral devices measured using a narrowband optical filter and extract the self-apodization function based on the at least one interferogram.

6. The spectral modeling system of claim 2, further comprising:
a characteristics extractor configured to receive measured spectra from at least one spectral device of the remaining spectral devices measured with variable temperature and extract the thermal drift based on the measured spectra.

7. The spectral modeling system of claim 1, further comprising:
a characteristics extractor configured to receive measured spectra of universal samples different than the plurality of samples from at least a portion of the plurality of spectral devices and extract the spectral device characteristics of the plurality of spectral devices using measured spectra.

8. The spectral modeling system of claim 7, wherein the at least the portion of the plurality of spectral devices comprises each of the plurality of spectral devices.

9. The spectral modeling system of claim 7, wherein the at least the portion of the plurality of spectral devices comprises selected spectral devices of the plurality of spectral devices having corresponding spectral device characteristics covering a space of variations including corners of production line characteristics of a production line comprising the plurality of spectral devices.

10. The spectral modeling system of claim 1, further comprising:
a characteristics extractor configured to generate the spectral device characteristics based on statistical information related to a production line comprising the plurality of spectral devices.

11. The spectral modeling system of claim 10, wherein the characteristics extractor is further configured to derive statistical parameters of the spectral device characteristics based on the statistical information.

12. The spectral modeling system of claim 11, wherein the statistical parameters comprise one or more of a mean value, standard deviation, skewness, or kurtosis.

13. The spectral modeling system of claim 12, wherein the characteristics extractor is further configured to determine a probability distribution of each of the statistical parameters and to generate the spectral device characteristics based on the statistical parameters and the respective probability distribution of each of the statistical parameters.

14. The spectral modeling system of claim 1, wherein the subset of the plurality of spectral devices comprises a single spectral device.

15. The spectral modeling system of claim 1, wherein the spectral converter is further configured to apply a spectral variance function to the spectral data to produce processed spectral data representative of variances in the subset of the plurality of spectral devices.

16. The spectral modeling system of claim 1, wherein the spectral converter is further configured to apply a spectral correction function to the spectral data to produce processed spectral data that removes uncontrolled variances in the subset of the plurality of spectral devices.

17. The spectral modeling system of claim 1, wherein the spectral converter is further configured to apply a spectral modulation and perturbation function to the spectral data to produce processed spectral data spanning different levels of aging and environmental conditions variations.

18. The spectral modeling system of claim 1, wherein the spectral converter is further configured to apply an optical head variance function to the spectral data to produce processed spectral data that accounts for different optical head configurations in the subset of the plurality of spectral devices.

19. The spectral modeling system of claim 1, wherein the spectral converter is further configured to apply a set of apodization functions to the spectral data to produce the plurality of artificial spectra.

20. The spectral modeling system of claim 1, wherein the spectral converter is further configured to add wavelength errors to the spectral data to produce the plurality of artificial spectra.

21. The spectral modeling system of claim 1, wherein the spectral converter is further configured to add noise across a spectral range corresponding to a signal-to-noise ratio (SNR) distribution to the spectral data to produce the plurality of artificial spectra.

22. The spectral modeling system of claim 1, wherein the spectral converter is further configured to scale an absorbance spectrum of the spectral data using a wavelength dependent scaling factor to produce the plurality of artificial spectra.

23. The spectral modeling system of claim 1, wherein the spectral converter is further configured to multiply the spectral data by a thermal drift factor across wavelength to produce the plurality of artificial spectra.

24. The spectral modeling system of claim 1, wherein the spectral converter is further configured to add baseline variations to absorbance of the spectral data to produce the plurality of artificial spectra.

25. The spectral modeling system of claim 1, wherein the spectral converter is further configured to add back reflection spectra to the spectral data to produce the plurality of artificial spectra.

26. The spectral modeling system of claim 25, wherein the spectral converter is further configured to multiply an Etalon effect to the spectral data to produce the plurality of artificial spectra.

27. The spectral modeling system of claim 1, wherein the spectral converter is further configured to multiply background material reflectance variations associated with background materials used to produce the spectral device characteristics to the spectral data to produce the plurality of artificial spectra.

28. The spectral modeling system of claim 1, wherein the spectral converter is further configured to apply optical path difference (OPD) errors to the spectral data to produce the plurality of artificial spectra.

29. The spectral modeling system of claim 1, wherein the spectral converter is further configured to optimize the spectral device characteristics based on measured values from test spectral devices of the plurality of spectral devices.

30. The spectral modeling system of claim 1, wherein the spectral converter is further configured to alter a distribution of the plurality of artificial spectra with respect to a corresponding measured value of the plurality of samples.

31. The spectral modeling system of claim 1, wherein the chemometrics engine is further configured to select the subset of the plurality of spectral devices, select one or more wavelength ranges for the spectral data, remove fluctuations in the spectral data resulting from improper measurement or variations in the subset of the plurality of spectral devices, and train the chemometrics model based on the spectral data and the plurality of artificial spectra.

32. The spectral modeling system of claim 31, wherein the chemometrics engine is further configured to adjust the chemometrics model using additional spectral data from deviant spectral devices of the plurality of spectral devices that deviate in performance from regular spectral devices of the plurality of spectral devices.

33. The spectral modeling system of claim 1, wherein the chemometrics engine is further configured to optimize a number of latent variables used to produce the chemometrics model to minimize a bias between test spectral devices of the remaining spectral devices and produce a root mean squared error within a specified range from a target minimum value.

34. The spectral modeling system of claim 1, wherein the subset of the plurality of spectral devices comprises multiple spectral devices, and wherein the chemometrics engine is further configured to identify a unified spectral dataset based on the spectral data by projecting the spectral data onto a space that is uncorrelated with a subspace of spectral device specification discrepancies.

35. The spectral modeling system of claim 1, wherein the subset of the plurality of spectral devices comprises multiple spectral devices, and wherein the chemometrics engine is further configured to form a matrix describing discrepancies between the subset of the plurality of spectral devices for each measurement in the spectral data and apply a conditional dimensionality reduction on the spectral data using the matrix.

36. The spectral modeling system of claim 1, wherein the spectral data comprises measurements of phantom samples corresponding to the plurality of samples, each of the phantom samples comprising a stable substance having a same absorbance spectra as one of the plurality of samples.

37. The spectral modeling system of claim 33, wherein the chemometrics engine is further configured to calibrate additional spectral devices using the phantom samples and the chemometrics model.

38. The spectral modeling system of claim 1, wherein the chemometrics engine is further configured to generate a transfer function using a set of samples measured on one or more of the plurality of spectral devices and a different spectral device comprising a different configuration than any of the plurality of spectral devices and generalize the chemometrics model to include the different spectral device based on the transfer function.

39. The spectral modeling system of claim 1, wherein the chemometrics engine is further configured to produce the chemometrics model for the plurality of samples based on the spectral data, the plurality of artificial spectra, and additional spectral device characteristics of the subset of the plurality of spectral devices.

40. The spectral modeling system of claim 39, wherein the chemometrics engine is further configured to receive a sample measurement of a sample under test from a test spectral device of the plurality of test devices, the sample under test corresponding to one of the plurality of samples, receive test spectral device characteristics of the test spectral device, and generate a result using the chemometrics model, the sample measurement, and the test spectral device characteristics.

41. The spectral modeling system of claim 39, wherein the chemometrics engine is a cloud-based artificial intelligence engine configured to store the chemometrics model and test spectral device characteristics and other test spectral device characteristics of other test spectral devices of the plurality of spectral devices.

42. The spectral modeling system of claim 1, wherein the spectral converter is further configured to access a library of pre-calculated stored transfer functions, select one or more selected transfer functions of the pre-calculated stored transfer functions based on the spectral device characteristics, and use the one or more selected transfer functions to generate the plurality of artificial spectra.

43. The spectral modeling system of claim 1, wherein the spectral converter is further configured to extract difference spectra between the plurality of artificial spectra and the spectral data, the difference spectra corresponding to clutter signals indicative of device variations between the plurality of spectral devices, and filter the clutter signals from the spectral data and the plurality of artificial spectra to produce filtered data used to generate the chemometrics model.

44. The spectral modeling system of claim 1, wherein the spectral converter is further configured to extract difference spectra between the plurality of artificial spectra and the spectral data, the difference spectra corresponding to a repeatability file indicative of device variations between the plurality of spectral devices, and wherein the chemometrics engine is further configured to use the repeatability file together with corresponding zero reference values to generate the chemometrics model.

45. The spectral modeling system of claim 1, wherein the spectral converter is further configured to receive a development dataset of a subset of the plurality of samples measured by a development kit comprising an additional subset of the plurality of spectral devices larger than the subset of the plurality of spectral devices, merge the spectral data and the development dataset to produce a merged dataset, and use the merged dataset to generate the plurality of artificial spectra.

46. The spectral modeling system of claim 1, wherein the chemometrics model is a cloud-based chemometrics model accessible to the plurality of spectral devices.

47. A method for spectral modeling, comprising:
receiving spectral data of a plurality of samples from a subset of a plurality of spectral devices;
receiving spectral device characteristics representing spectral variations in the plurality of spectral devices;
generating a plurality of artificial spectra representing remaining spectral devices of the plurality of spectral devices based on the spectral data and the spectral device characteristics; and
producing a chemometrics model for one or more parameters associated with the plurality of samples based on the spectral data and the plurality of artificial spectra.

48. The method of claim 47, further comprising:
receiving measured spectra of universal samples different than the plurality of samples from at least a portion of the plurality of spectral devices; and
extracting the spectral device characteristics of the plurality of spectral devices using measured spectra.

49. The method of claim 47, further comprising:
accessing a library of pre-calculated stored transfer functions;
selecting one or more selected transfer functions of the pre-calculated stored transfer functions based on the spectral device characteristics; and
using the one or more selected transfer functions to generate the artificial spectra.

50. The method of claim 47, further comprising:
extracting difference spectra between the plurality of artificial spectra and the spectral data, the difference spectra corresponding to clutter signals indicative of device variations between the plurality of spectral devices; and
filtering the clutter signals from the spectral data and the plurality of artificial spectra to produce filtered data used to generate the chemometrics model.

51. The method of claim 47, further comprising:
extracting difference spectra between the plurality of artificial spectra and the spectral data, the difference spectra corresponding to a repeatability file indicative of device variations between the plurality of spectral devices; and
using the repeatability file together with corresponding zero reference values to generate the chemometrics model.
